(19) United States
(12) Patent Application Publication
(10) Pub. No.: US 2019/0271693 A1
(43) Pub. Date: Sep. 5, 2019

(54) METHODS, COMPOSITIONS, AND KITS FOR DETECTING BACTERIOPHAGE IN A SAMPLE

(71) Applicant: Sonanutech Inc., Davis, CA (US)

(72) Inventors: Sudheendra Lakshmana, Davis, CA (US); Laura Nicole Putnam, Davis, CA (US); Alice Ngo, West Sacramento, CA (US); Alexander Thornton-Dunwoody, Davis, CA (US); Leslie Anne Jones, Davis, CA (US)

(73) Assignee: Sonanutech Inc., Davis, CA (US)

(21) Appl. No.: 16/462,235

(22) PCT Filed: Nov. 24, 2017
(86) PCT No.: PCT/US17/63196

§ 371 Date: May 19, 2019

Related U.S. Application Data
(60) Provisional application No. 62/427,084, filed on Nov. 28, 2016.
(60) Provisional application No. 62/450,361, filed on Jan. 25, 2017.
(60) Provisional application No. 62/453,099, filed on Feb. 1, 2017.
(60) Provisional application No. 62/453,099, filed on Feb. 1, 2017.
(60) Provisional application No. 62/518,600, filed on Jun. 13, 2017.
(60) Provisional application No. 62/518,598, filed on Jun. 13, 2017.

(51) Int. Cl. G01N 33/543; C12Q 1/04; G01N 33/569; C12Q 1/70; G01N 27/327; G01N 33/04; C12N 7/00

(52) U.S. Cl. CPC G01N 33/5438; C12Q 1/04; G01N 33/56911; C12N 7/00; G01N 27/327; G01N 33/04; C12Q 1/70

(57) ABSTRACT

Provided are methods, compositions, devices, and kits for detecting bacteriophage contamination in a sample. The methods, compositions, devices, and kits are specially useful when the identity of the bacteriophage is unknown. The methods, compositions, devices, and kits are applicable to a wide variety of industries that employ bacterial fermentation, for example, dairy and wine industries.

### CROSS-REFERENCE TO RELATED APPLICATIONS

This application claims the benefit of U.S. Provisional Patent Application Ser. No. 62/427,084 filed Nov. 26, 2016; U.S. Provisional Patent Application Ser. No. 62/450,361 filed Jan. 25, 2017; U.S. Provisional Patent Application Ser. No. 62/453,099 filed Feb. 1, 2017; U.S. Provisional Patent Application Ser. No. 62/518,598 filed Jun. 13, 2017; and U.S. Provisional Patent Application Ser. No. 62/518,600 filed Jun. 13, 2017, the entire contents of each of which are incorporated by reference herein.

### STATEMENT REGARDING FEDERALLY SPONSORED RESEARCH AND DEVELOPMENT

The invention was made with Government support under. Grant Nos. 1534756 awarded by the National Science Foundation. The government has certain rights in the invention.

### FIELD OF THE INVENTION

Provided are methods, compositions, devices, and kits for detecting bacteriophage contamination in a sample.

### BACKGROUND OF THE INVENTION

Contamination with phages results in significant economic impact from loss of raw material and downtime due to the need to sanitize and disinfect a facility thoroughly. Lytic phages are a significant challenge for any industry relying on bacterial fermentation, e.g., dairy industry, wine industry, meat and vegetable fermentation industries, and vinegar industry.

In a dairy industry, to generate one ton of cheese, about 10¹⁴ active microbes are required. Although the phage concentration in milk and microbial culture is low, the phages can amplify significantly in number, reaching 10¹²-10¹⁸ phages in a 1000 to 10000 L bioreactor that is used to prepare culture blends, if susceptible microbial cells are present. This amplification of phages results in a compromise of the host microbes and failure of the fermentation process. Phage contamination can result from both lytic and lysogenic phages. Compared to lysogenic phages, lytic phages are the major cause of loss in microbial fermentation processes (over 90%). Although the presence of lysogenic or prophages in microbial cultures can also induce generation of active phages during fermentation, the cheese industry has adopted screening methods to select culture strains with significant resistance to activation of prophages during microbial fermentation. Lytic phages belonging to Siphoviridae and Podoviridae families are mainly responsible for milk fermentation failures worldwide. These species are highly robust and can survive in fermentation facilities despite best practice and process control protocols.

In an industrial setting, two methods for detection of phages are followed: 1) the Heap-Lawrence test, which is based on acidification rate of milk and gives a reliable, albeit not unique, indication of phage infection (other factors may lead to reduced acidification); 2) a plaque-forming assay, which allows quantitative analysis of strain-specific phage counts. Both methods can take from 1 to 2 days for the detection of phage infection, although the plaque-forming assay is highly specific and is the gold standard for phage detection and classification. Faster and more sensitive methods of detecting a phage infection early in the process are highly sought-after by the cheese industry.

Bacteriophages evolve by changing their DNA. This, in many cases, changes their binding domains in the tail of the phages and hence their host bacteria. A RT-PCR based assay detects only a narrow class of phages whose genetic information is known. Immunoassay can be used to detect bacteriophages by using the antibody to a specific antigen if such antigen is known and present. Therefore, RT-PCR and immunoassays have limited detection ability towards wild-type phages.

### SUMMARY OF THE INVENTION

In one aspect, provided herein are methods of detecting one or more types of bacteriophage in a sample. The methods include a) providing a first sample suspected of comprising one or more types of bacteriophage, b) providing a second sample comprising one or more types of bacteria susceptible to infection by one or more types of bacteriophage, c) contacting the first sample with the second sample to form a sample mixture comprising one or more complexes of bacteria and bacteriophage, if bacteriophage is present in the sample, d) contacting the sample mixture with one or more compounds, e) measuring any change of one or more compounds in presence of the sample mixture. A change of one or more compounds is indicative of the presence of one or more types of bacteriophage infected bacteria in the sample mixture, and the presence of one or more types of bacteriophage infected bacteria in the sample mixture is indicative of the presence of one or more types of bacteriophage in the first sample.

In some embodiments, the change of one or more compound is measured over time and the method further includes determining the kinetic profile of the change of one or more compounds. The kinetic profile is indicative of indicative of the presence of one or more types of bacteriophage infected bacteria in the sample mixture, and the presence of one or more types of bacteriophage infected bacteria in the sample mixture is indicative of the presence of one or more types of bacteriophage in the first sample.

In some embodiments, the method includes comparing the kinetic profile of the change of one or more compounds from the sample mixture with a kinetic profile of the change of one or more compounds in the presence of uninfected bacteria. A change of the kinetic profile is indicative of the presence of bacteriophage in said sample.

In some embodiments, the identity of the bacteriophage is unknown. In some embodiments, one or more compounds undergo a change in the oxidative state. In some embodiments, one or more compounds undergo reduction. In some embodiments, the compounds undergo oxidation. In some embodiments, one or more compounds undergo oxidation and/or reduction. In some embodiments, the oxidation and/or reduction of compounds is reversible. In some embodiments, the oxidation and/or reduction of the compounds is irreversible. In some embodiments, one or more compounds are enzymatic substrates of said one or more bacteria.

In some embodiments, the compound is Resazurin, Resorufin, Dihydroresorufin, and/or a combination of these compounds. In some embodiments, the Resazurin is reduced to Resorufin. In some embodiments, the Resazurin (weakly fluorescent) gets reduced to fluorescent Resorufin due to metabolic activity of the cell. This reduction is irreversible. In some embodiments, Resorufin is further reduced by the cell to Dihydroresorufin (non-fluorescent). This reduction is reversible. The inventors have surprisingly and unexpectedly found that the equilibrium between Resorufin and Dihydroresorufin is significantly affected by the presence of phage in the sample. In the case of phage infected cells, the Dihydroresorufin is preferably oxidized back to Resorufin, while in the case of uninfected cells the Dihydroresorufin is a favored product in the sample mixture. In some embodiments, in the case of phage infected cells, the inventors have surprisingly and unexpectedly found that the formation of Dihydroresorufin is not favored when the phage concentration is high. Also, inventors of the present application have surprisingly and unexpectedly found that in the case of phage infected cells, the reduction of Resazurin to Resorufin is slower than that of the uninfected cells. Thus, the fluorescence intensity and/or absorbance intensity of Resorufin appears faster for non-infected cells as compared to phage infected cells. In some embodiments, in the case of phage infected cells, the fluorescent intensity and/or absorbance intensity of Resorufin, does not decrease as reduction to Dihydroresorufin is not favored. In some embodiments, the fluorescent intensity and/or absorbance intensity of Resorufin increases with time in comparison to Dihydroresorufin as Dihydroresorufin is oxidized back to Resorufin.

In contrast, in the case of uninfected cells the fluorescent and/or absorbance intensity of Resorufin diminishes with time as Resorufin is further reduced to Dihydroresorufin and does not reappear or reappears at a significantly later time than in the case of phage infected cells.

The inventors of the present application have employed this unexpected finding in detecting the presence of bacteriophage in a sample. In some embodiments, detection includes determining the change in the color, color intensity, light scattering, fluorescence and/or absorbance intensity, change in the slope of the fluorescence and/or absorbance kinetic profile of Resazurin to Resorufin and/or Resorufin to Dihydroresorufin reduction by the bacteriophage infected bacteria if the phage is present in the sample.

In some embodiments, the methods further include comparing the kinetic profile of Resazurin reduction by the sample with that of uninfected bacteria (control) in which a shift in the kinetic peak is indicative of the presence of one or more types of bacteriophage infected bacteria and hence one or more types of bacteriophage in the sample. Additionally, the diminution of the Resorufin, as measured by change in color, light scattering, fluorescence and/or absorbance intensity with time and reappearance of its fluorescence intensity is indicative of phage infected bacteria and thus indicative of the phage in a sample. Further, no diminution and/or slow diminution of the Resorufin as measured by change in color, light scattering, fluorescence and/or absorbance intensity with time and reappearance of its fluorescence intensity is indicative of phage infected bacteria and thus indicative of the phage in a sample. In contrast, a diminution of the Resorufin fluorescent intensity without its reappearance in a significant amount of time is indicative of uninfected bacteria and thus absence of bacteriophage in a sample.

In some embodiments, one or more types of bacterial cells are susceptible to infection by one or more types of bacteriophage are incubated with samples suspected of comprising one or more types of bacteriophage to form one or more types of complexes if one or more types of bacteriophage is present in a sample, prior to adding Resazurin to the incubation mixture. In some embodiments, Resazurin is replaced by adding Resorufin, Dihydroresorufin, or a combination of the compounds to the incubation mixture. In some embodiments, the bacterial cells susceptible to infection by the bacteriophage are incubated with samples suspected of comprising bacteriophages for at least about 1, 2, 3, 5, 7, 10, 15, 20, 25, 30, 35, 40, 45, 50, 60, 65, 70, 75, 80, 90 or more minutes prior to adding Resazurin, Resorufin, Dihydroresorufin or a combination of these compounds to the incubation mixture. In some embodiments, the bacterial cells susceptible to infection by the bacteriophage and the samples suspected of comprising bacteriophages are incubated together with Resazurin and/or Resorufin and/or Dihydroresorufin. In some embodiments, the incubation is carried out at about 20° C., 25° C., 27° C., 30° C., 32° C., 33° C., 34° C., 35° C., 36° C., 37° C., 40° C., 42° C., 43° C., 44° C., 45° C., or more.

In some embodiments, the fluorescence of Resorufin is measured between 530-650 nm. In some embodiments, the fluorescence of Resorufin is measured at about 550 nm, 555 nm, 560 nm, 564 nm, 570 nm, 572 nm, 573 nm, 575 nm, 577 nm, or 580 nm or higher wavelength. In some embodiments, the fluorescence of Resorufin is measured for about 2, 5, 10, 15, 20, 25, 30, 35, 40, 45, 50, 55, 60, 65, 70, 75, 80, 85, 90, 95, 100, 105, 110, 115, 120, 125, 130, 135, 140 or more minutes after adding Resazurin, Resorufin, Dihydroresorufin or a combination of these compounds to the incubation mixture of bacteria and samples suspected of comprising bacteriophages.

In some embodiments, the methods comprise simultaneously detecting different types of bacteriophages in the same assay. In some embodiments, the methods are multiplexed. In some embodiments, methods include simultaneously using different types of bacteria in which each of such bacteria are susceptible to a particular type of bacteriophage.

In one aspect, provided herein are kits for detecting the presence of one or more types of bacteriophage in a sample. The kits include a) a first reagent comprising one or more types of bacteria in which the one or more types of bacteria are susceptible to infection by the one or more types of bacteriophage, and the one or more types of bacteria forms one or more types of complexes with one or more types of bacteriophage if present in the sample; b) buffers; c) a second reagent comprising one or more compounds. The one or more compounds are differentially susceptible to change in the presence of phage infected and uninfected bacteria; d) instruction for forming one or more complexes of the bacteria with the bacteriophage; and e) instruction for detecting any change to one or more compounds. A differential change in one or more compounds for phage infected as compared to uninfected bacteria is indicative of the presence of said bacteriophage in the sample.

In some embodiments, the one or more compounds of the second reagent of the kit comprises a detectable label. In some embodiments, the one or more compounds of the second reagent comprise a redox compound. In some embodiments, the one or more compounds of the second reagent comprises a fluorescent label. In some embodiments, the second reagent is Resazurin, Resorufin, Dihydroresorufin or a combination of these compounds. In some embodiments, the kit further comprises a device for detection of the change of one or more compounds. In some embodiments, the device comprises a fluorescence detector. In some embodiments, the device comprises an electrochemical detector. In some embodiments, the device comprises a fluidic chamber in which the chamber allows a fluid communication of the one or more types of bacteriophage if present in the sample and the one or more types of bacteria in the first reagent.

In one aspect, provided herein are devices for detecting the presence of one or more types of bacteriophage in a sample. The devices include a) a first fluidic chamber for accepting a first sample suspected of comprising one or more types of bacteriophage; b) a second fluidic chamber for accepting a second sample comprising one or more types of bacteria. The one or more types of bacteria are susceptible to infection of the one or more types of bacteriophage, and the one or more types of bacteria form complexes with the one or more types of bacteriophage. The second fluidic chamber is in fluid communication with said first chamber; c) a fluidic means for contacting the complexes of the bacteria and the bacteriophage with one or more compound; d) a sensor for detecting any change of the compound; wherein a differential change of the compound in the presence of the complex as compared to uninfected bacteria is indicative of the presence of the bacteriophage in the sample.

In some embodiments, the one or more compounds comprise one or more redox compounds. In some embodiments, the redox compounds are Resazurin, Resorufin, Dihydroresorufin, or a combination of one or more of these compounds. In some embodiments, the sensor is a fluorescent sensor. In some embodiments, the sensor is an electrical and/or electrochemical sensor. In some embodiments, the fluorescence sensor has both excitation and detection elements. In some embodiments, the device has a microchip. In some embodiments, the device further comprises a thermostat. In some embodiments, the device further comprises a heating element.

In some embodiments, the device comprises a mechanical slide. In some embodiments, the device comprises of a mechanical motor. In some embodiments, the motor is attached to a stage. In some embodiments, the stage has a block. In some embodiments, the block has holes to insert sample tubes. In some embodiments, block and/or the samples in the sample tubes are heated by a heating element. In some embodiments, the heater is regulated to maintain the temperature of the block and/or the samples. In some embodiments, the block and/or samples in the sample tubes are moved under the fluorescence sensor. In some embodiments, the fluorescence sensor collects the fluorescence from the samples sequentially. In some embodiments, the fluorescence is performed in epifluorescence method. In some embodiments, the fluorescence is detected in confocal method. In some embodiments, the fluorescence is detected in an off-axis mode. In some embodiments, the fluorescence signals collected from the samples are processed by microprocessor. In some embodiments, the processed signal from the microprocessor is displayed on the digital screen. In some embodiments, the microprocessor controls the functioning of the heater. In some embodiments, the microprocessor controls the functioning of the thermostat. In some embodiments, the microprocessor controls the motor. In some embodiments, the microprocessor controls the display unit.

In one aspect, provided herein are methods for detecting the presence of bacteriophage in a sample, whose genetic information is either known or unknown. The methods include a) providing a first sample suspected of having the bacteriophage; b) providing a second sample having bacteria, in which the bacteria are susceptible to the bacteriophage, and c) incubating the first sample with the second sample in which the bacteriophage if present in the first sample forms a complex with the bacteria. The complex of the bacteriophage and the bacteria is detected. The detection of the complex is indicative of the presence of said bacteriophage in the first sample.

In one aspect, provided herein are methods for detecting the presence of bacteriophage in a sample. The methods include a) providing a first sample suspected of having the bacteriophage; b) providing a second sample having bacteria, in which the bacteria are susceptible to the bacteriophage, and c) incubating the first sample with the second sample in which the bacteriophage if present in the first sample forms a complex with the bacteria. The complex is separated from the incubation mixture comprising bacteriophage and bacteria. The nucleic acid from the bacteriophage and the bacteria of the complex are isolated. The bacteriophage nucleic acid is selectively amplified. The selective amplification of the bacteriophage nucleic acid is indicative of the presence of the bacteriophage in said sample. In some embodiments, blunt ends of isolated bacteriophage nucleic acid are created and identifier sequences are ligated to the blunt ends. The identifier sequence comprises a unique primer binding site. The isolated bacteriophage nucleic acid is amplified using said identifier sequence.

In one aspect, provided herein are methods of detecting bacteriophage contamination in a sample. The methods include a) providing a sample comprising a complex of bacteriophage and bacteria susceptible to infection by said bacteriophage, b) isolating nucleic acid from the bacteriophage, c) selectively amplifying the nucleic acid from the bacteriophage in which selective amplification of the bacteriophage nucleic acid is indicative of the presence of bacteriophage in the sample.

In some embodiments, the methods further include creating blunt ends of isolated bacteriophage nucleic acid and ligating identifier sequences to the blunt ends in which the identifier sequence comprises a unique primer binding site. The isolated bacteriophage nucleic acids are amplified using said identifier sequence.

In one aspect, provided are devices for detecting the presence of bacteriophage in a sample. The devices include a) a solid support in which the solid support includes one or more functional groups for immobilizing a bacteriophage or bacteria susceptible to infection by the bacteriophage and b) a sensor. The sensor is capable of detecting a complex of the bacteriophage and the bacteria. In some embodiments, the device further includes fluid channel for fluid communication between immobilized bacteriophage or bacteria with its counterpart. Exemplary functional groups include, but are not limited to functional group is selected from the group consisting of poly L-lysine, aminosilane, epoxysilane, aldehydes, carboxylic groups, azide, alkalyne, maleimide, amino groups, epoxy groups, cyano groups, ethylenic groups, hydroxyl groups, thiol groups, and N-hydroxysuccinimide(NHS). In some embodiments, the solid support of the device includes metal coated surface. In some embodiments, the solid support of the device includes dielectric coated glass. In some embodiments, the solid support of the device includes nanowells. In some embodiments, the solid support of the device includes microarray. In some embodiments, the device includes a fluidic chamber. In some embodiments, the fluidic chamber of the device includes a second metal coated surface.

In one aspect, provided are kits for detecting the presence of bacteriophage in a sample. The kits include a) a reagent having bacteria, which are susceptible to the infection by the bacteriophage; b) buffers useful for detection of the bacteriophage; c) instruction for forming a complex of the bacteria with the bacteriophage; d) instruction for detecting the complex. Detecting the complex is indicative of the presence of said bacteriophage in the sample. In some embodiments, the kits further include one or more detectable labels. The detectable labels associate with the protein or nucleic acid of the bacteria or the bacteriophage. In some embodiments, the kits further include a device for detection of the complex. In some embodiments, the device includes a sensor. The sensor is capable of detecting the complex. In some embodiments, the device includes a fluidic chamber in which the chamber allows a fluid communication of the bacteriophage and the bacteria. In some embodiments, the device includes a solid support capable of immobilizing the bacteria and/or the bacteriophage. In some embodiments, the solid support includes one or more functional groups for immobilizing the bacteriophage and/or the bacteria. Exemplary functional groups include, but are not limited to functional group is selected from the group consisting of poly L-lysine, aminosilane, epoxysilane, aldehydes, carboxylic groups, azide, alkalyne, maleimide, amino groups, epoxy groups, cyano groups, ethylenic groups, hydroxyl groups, thiol groups, and N-Hydroxysuccinimide (NHS), streptavidin, azide, alkyne and maleimide.

In some embodiments, the identity of said bacteriophage is unknown. In some embodiments, the genetic information of the bacteriophage is unknown. In some embodiments, the taxonomy of the bacteriophage is unknown.

In some embodiments, the sample suspected of being contaminated by one or more types of bacteriophage can be water, liquid collected from facility equipment, swabbed facility surfaces, fluid samples from facilities, fermented liquid, raw milk, pasteurized milk, skim milk, whey, milk starter culture, whey starter culture and broth grown starter culture.

In some embodiments, the sample suspected of being contaminated by one or more types of bacteriophage is partitioned into solid phase and liquid phase prior to incubating with a sample comprising one or more types of bacteria in which the partitioned liquid phase of the sample is incubated with the sample comprising one or more types of bacteria. In some embodiments, the partitioning of the sample into solid and liquid phases is by centrifugation. In some embodiments, the partitioning of the sample into solid and liquid phases is by filtration. In some embodiments, the partitioning of the sample into solid and liquid phases is by lowering the pH. In some embodiments, the partitioning of the sample into solid and liquid phases is by a combination of lowering the pH and filtration. In some embodiments, the partitioning of the sample into solid and liquid phases is by a combination of lowering the pH and centrifugation. In some embodiments, the partitioning of the sample into solid and liquid phases is by a combination of lowering the pH, centrifugation, and filtration.

In some embodiments, the bacteriophage in the sample is concentrated prior to incubating with another sample comprising bacteria. In some embodiments, the concentration of the bacteriophage is by changing the pH. In some embodiments, the concentration of the bacteriophage is by salt concentration. In some embodiments, the concentration of the bacteriophage is by centrifugation. In some embodiments, the concentration of the bacteriophage is by filtration. In some embodiments, the filtration is by membrane filtration. In some embodiments, the pore size of the membrane is about 1 nm to about 200 nm. In some embodiments, the concentration of the bacteriophages is by changing the ionic strength of the sample. In some embodiments, the concentration of the bacteriophages is by changing the pH of the sample. In some embodiments, the methods further include partitioning the first sample into solid and liquid phases.

In some embodiments, the methods further include separating at least a portion of the complex of the bacteriophage and the bacteria from the incubation mixture prior to detection of the complex. In some embodiments, the separation of the complex is by applying an electric field. In some embodiments, the separation of the complex is by size. In some embodiments, the separation of the complex is by centrifugation. In some embodiments, the separation of the complex is by filtration. In some embodiments, the filtration is by membrane filtration. In some embodiments, the pore size of the membrane is about 1 nm to about 1 Aim. In some embodiments, the separation of the complex is based on the difference in density of the complex, the bacteria, and the bacteriophage.

In some embodiments, the bacteria susceptible to bacteriophage infection are immobilized on a solid support. In some embodiments upon incubation of the sample comprising bacteriophage with another sample comprising bacteria which are susceptible to the infection by the bacteriophage and which are immobilized on a solid support, at least a portion of the bacteriophage in the sample attach to specific receptors of the immobilized bacteria to form a complex of bacteriophage and bacteria. Accordingly, at least a portion of said complexes are immobilized on the solid support. In some embodiments, at least a portion of said complex immobilized on the solid support is separated from the incubation mixture of the samples comprising bacteriophage and bacteria prior to detection of said complex.

36. In some embodiments, the bacteriophage in the sample is immobilized on a solid support. In some embodiments upon incubation of the sample comprising bacteriophage with another sample comprising bacteria which are susceptible to the infection by the bacteriophage, at least a portion of the bacteriophage in the sample attach to specific receptors of the bacteria to form a complex of bacteriophage and bacteria in which at least a portion of the complexes are immobilized on the solid support through the immobilized bacteriophage.

In some embodiments, at least a portion of the complex immobilized on the solid support is separated from the incubation mixture of the samples comprising bacteriophage and bacteria prior to detection of the complex.

In some embodiments, the solid support comprises a first member of a binding pair and said bacteria or bacteriophage to be immobilized on the solid support comprises a second member of the binding pair. Binding of the first and second members of the binding pair immobilizes the bacteria or bacteriophage to the solid support. In some embodiments, the binding pair is biotin and streptavidin.

In some embodiments, the bacteria or bacteriophage is immobilized on said solid support by covalent bond. In some embodiments, the solid surface comprises functional groups for immobilization by covalent bond. Non-limiting examples of functional group include poly L-lysine, aminosilane, epoxysilane, aldehydes, carboxylic groups, azide, alkalyne, maleimide, amino groups, epoxy groups, cyano groups, ethylenic groups, hydroxyl groups, thiol groups, and N-Hydroxysuccinimide (NHS).

In some embodiments, the solid support is a bead. In some embodiments, the bead is a magnetic bead. In some embodiments, the bead is a streptavidin bead. In some embodiments, the bead comprises antibody. In some embodiments, the solid support is a microarray. In some embodiments, the solid support is a fluidic device. In some embodiments, the solid support is a flow cell. In some embodiments, the solid support is a fluidic cartridge. In some embodiments, the solid support comprises metal coated surface. In some embodiments, the solid support comprises dielectric coated glass. In some embodiments, the solid support comprises nanowells, nanochannels and/or nanostructures. In some embodiments, the solid support is a porous material. In some embodiments, the solid support comprises a sensor.

In some embodiments, the bacteriophages or the bacteria comprise a detectable label and the complex is detected by detecting the detectable label. In some embodiments, the bacteriophages and the bacteria comprise detectable labels and the complex is detected by detecting the detectable labels. In some embodiments, the detectable labels of the bacteriophages and the bacteria are same. In some embodiments, the detectable labels of the bacteriophage and the bacteria are different. In some embodiments, the detectable labels are present on the surface of the bacteria or the bacteriophage. In some embodiments, the detectable labels are present inside the bacteria or the bacteriophage.

In some embodiments, the bacteriophage comprises more than one detectable label. In some embodiments, the bacteriophage can comprise at least two detectable labels. In some embodiments, the detectable labels are different. In some embodiments, one detectable label is associated with the nucleic acid of the bacteriophage and a second detectable label is associated with the bacteriophage proteins.

In some embodiments, the bacteria comprise more than one detectable label. In some embodiments, the bacteria can comprise at least two detectable labels. In some embodiments, the detectable labels are different. In some embodiments, one detectable label is associated with the nucleic acid of the bacteria and a second detectable label is associated with the bacterial proteins.

In some embodiments, the detectable label is associated with the nucleic acid or the protein of the bacteriophages and/or the bacteria. In some embodiments, the detectable label comprises an antibody specific for a protein of said bacteriophage. In some embodiments, the detectable label comprises an antibody specific for a protein of said bacteria.

In some embodiments, the detectable label comprises a fluorescent moiety. Non-limiting examples of fluorescent moiety include cyanine and non-cyanine based dyes such as TOTO®, YOYO®, BOBO™, POPO™, Propidium Iodide and its derivatives, PMA™ (propidium monoazide), SYBR®, SYTOX®, Cy2, Cy3, Cy3.5, Cy5, Cy5.5, Cy7, Alexa Fluor® 350, Alexa Fluor® 405, Alexa Fluor® 430, Alexa Fluor® 488, Alexa Fluor® 532, and Alexa Fluor® 632. In some embodiments, the detection of the complex is by detecting the fluorescence signal from said fluorescent moiety.

In some embodiments, the detectable label is associated with the bacteria or the bacteriophage by van der Waals interaction, electrostatic interaction, hydrogen boding interactions, magnetic interaction, covalent interaction, or a combination thereof.

In some embodiments, the complex is detected by Raman spectroscopy. In some embodiments, the complex is detected by surface enhanced Raman spectroscopy. In some embodiments, the illuminating laser has a wavelength of about 300 nm to about 2000 nm. In one embodiment, the illuminating laser has a wavelength of about 532 nm.

In some embodiments, the bacteria or said bacteriophage is immobilized on a solid support in which the solid support comprises a sensor. Upon incubation of the samples comprising bacteriophage and bacteria, at least a portion of the complexes thus formed are immobilized on the solid support. In some embodiments, the complex is detected by a change in mass on the solid support. In some embodiments, the complex is detected by change in resonance frequency of the sensor. In some embodiments, the solid support comprises piezoelectric sensor in which the complex is detected by change in dissipation of shear movement of the sensor. In some embodiments, the complex is detected by detecting surface plasmon resonance. In some embodiments, the complex is detected by scanning probe microscopy. In some embodiments, the complex is detected by atomic force microscopy. In some embodiments, the complex is detected by electron microscopy. In some embodiments, the solid support comprises silicon nitride.

In some embodiments, the incubation is by fluid communication between said bacteria and said bacteriophage. In some embodiments, the solid support further comprises a flow cell in which the bacteria or the bacteriophage not immobilized on said solid support is in a fluid medium and the fluid medium is passed over the flow cell.

In some embodiments, the sensor comprises electrodes in which the complex is detected by a change in electrical signal. In some embodiments, voltage sensitive detectable label is used in which a complex is detected by a change in electrical signal once the complex is formed.

In some embodiments, the complex is detected by detecting the nucleic acid of the bacteriophage. In some embodiments, the detection of bacteriophage nucleic acid is by mass spectrometry. In some embodiments, the complex is detected by detecting the change in heat flow due to the formation of said complex. In some embodiments, the detection is by a calorimeter. In some embodiments, the calorimeter is an isothermal calorimeter. In some embodiments, the complex is detected by detecting the forward and sideward scattering of the light by the formation of said complex. In some embodiments, the detection is by a flow cytometer. In some embodiments, the complex is fluorescently stained. In some embodiments, the complex is detected by electrophoresis of the complex. In some embodiments, electrophoresis is done by extracting the DNA. In some embodiments, the electrophoresis can be gel pulse electrophoresis. In some embodiments, the information about the phage infection is deduced by looking at a DNA band in the range 20,000-200000 bps.

In some embodiments, the bacteria susceptible to bacteriophage infection are used in dairy fermentation, malolactic fermentation in wine making, in meat fermentation, vegetable fermentation, food fermentation, vinegar fermentation.

Exemplary list of bacteria used in dairy fermentation that is susceptible to bacteriophage infection includes, but is not limited to Lactococcus lactis ssp. cremoris; Lactococcus lactis ssp. lactis biovar diacetylactis; Lactococcus lactis ssp. lactis; Leuconostoc mesenteroides ssp. cremoris; Lactobacillus acidophilus; Lactobacillus delbrueckii ssp. bulgaricus; Lactobacillus delbrueckii ssp. lactis; Lactobacillus delbrueckii ssp. delbrueckii; Lactobacillus helveticus; Lactobacillus casei; Lactobacillus paracasei; Lactobacillus curvatus; Lactobacillusfermentum; Lactobacillus crispatus; Lactobacillus jensenii; Lactobacillus johnsonii; Lactobacillus rhamnosus; Leuconostoc mesenteroides ssp. cremoris; Bifidobacterium bifidus; Bifidobacterium animalis; Streptococcus thermophilus; Brevibacterium linens coryneform bacteria; Propionibacterium Acidipropionici and Propionibacterium freudenreichii ssp. shermanii.

Exemplary bacteria used in malolactic fermentation in wine making include bacteria lactic acid bacteria and Oenococcus oeni. Non-limiting examples of the genera of lactic acid bacteria includes Lactobacillus, Leuconostoc, Pediococcus, Lactococcus, and Streptococcus, Bifidobacterium, Aerococcus, Carnobacterium, Enterococcus, Oenococcus, Sporolactobacillus, Tetragenococcus, Vagococcus, and Weissella.

Exemplary list of bacteria used in meat fermentation includes, but are not limited to Lactobacillus curvatus, Lactobacillus sake, Pediococcus acidilactici, Pediococcuspentosaceus, Lactobacillus plantarum, Micrococcus varians, Staphylococcus carnosus, and Staphylococcus xylosus.

Exemplary list of bacteria used in acetic acid fermentation and vinegar production includes, but are not limited to Acetobacter aceti, Acetobacterfabarum, Acetobacter oeni, Acetobacter pomorum.

In some embodiments, the bacteria susceptible to bacteriophage infection are lactic acid bacteria (LAB) used in the conversion of lactose to lactic acid. In some embodiments, the LAB belongs to the genera of Aerococcus, Alloiococcus, Atopobium, Bifidobacterium, Carnobacterium, Enterococcus, Lactobacillus (Lb.), Lactococcus (L.), Leuconostoc (Leuc.), Oenococcus, Pediococcus, Streptococcus (S.), Tetragenococcus, Vagococcus and Weissella (W.)

Exemplary families of bacteriophage capable of infecting bacteria used in dairy and wine fermentation include but are not limited to Myoviridae, Siphoviridae and Podoviridae.

In some embodiments, the detection of a bacteriophage and bacteria complex is indicative of the presence of bacteria in a sample. In some embodiments, the sample is a fermentation sample. In some embodiments, the fermentation sample is a yeast fermentation sample and the bacteria is lactic acid bacteria. In some embodiments, the sample is a food. In some embodiments, the sample can be from a patient suspected of having a bacterial infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A depicts various forms of virulent bacteriophage that can infect the bacteria in a sample. FIG. 1B depicts various forms of non-virulent bacteriophage that cannot infect the bacteria in a sample.

FIG. 2 depicts various forms of bacteria that are susceptible to the bacteriophage present in a fermentation sample.

FIG. 3 depicts various forms of bacteria that are not susceptible to the bacteriophage present in a fermentation sample.

FIGS. 4 A-P depict various forms of complexes formed between the bacteriophage and the bacteria.

FIG. 5 shows exemplary solid supports.

FIG. 6 shows a cross section of an exemplary fluidic device.

FIG. 7 shows a schematic of selective amplification of bacteriophage DNA.

FIG. 8 shows the results of β-galactosidase activity of bacteriophage infected lactic acid bacteria

FIG. 9 shows the results of Resazurin to Resorufin, Live Cell Assay

FIG. 10 shows the results of the bacteriophage adhesion assay.

FIG. 11 shows the results of bacteriophage adhesion assay on microscopic slides.

FIG. 12A shows the need for critical concentration of bacterial cells to observe the oxidation of Dihydroresorufin to Resorufin. 12B-D, shows the effect of phage infection on cells below the critical bacterial cell concentration for different types of bacteria.

FIG. 13A-B shows the effect of temperature on the limit of detection of phage infection for a given cell concentration.

FIG. 14A-B shows the effect of initial redox reagent on the phage infection of the bacterial cells. In FIG. 14A, the initial redox reagent was Resazurin. In FIG. 14B, the initial redox reagent was Resorufin.

FIG. 15 shows the data of identifying a phage infection from a bacterial culture containing more than one species and genera.

FIG. 16 shows the data on difference in the kinetic profiles of phage infection when fresh cells and frozen cells are used.

FIG. 17 shows the fluorescence image of SYBR® Green dye (Row i) stained S1 bacteria added to different beads shown in the schematic (A-D). Row ii is the bright field image of the corresponding beads in Row i.

FIG. 18 shows the fluorescence spectra of SYTOX® Orange and Propidium Iodide.

FIGS. 19 A and 19B show the rendering of the detector

FIG. 20 shows the image of the detector.

### DETAILED DESCRIPTION OF THE INVENTION

The term “surface plasmon resonance,” as used herein, refers to an optical phenomenon that allows for the analysis of real-time biospecific interactions by detection of alterations in concentrations within a biosensor matrix, for example using the BIACORE™ system (Pharmacia Biosensor AB, Uppsala, Sweden and Piscataway, N.J.).

As used herein the term “at least a portion” and/or grammatical equivalents thereof can refer to any fraction of a whole amount. For example, “at least a portion” can refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 99.9% or 100% of a whole amount.

As used herein the term “about” means +/−10%.

As used herein the term “change” or “modification” of a compound refers to the change in the physical, chemical, or oxidative state of the compound. In some embodiments, the change comprises covalent modification. In some embodiments, a compound comprising a detectable label is modified upon association with bacteria or bacteriophage. In some embodiments, the modification is by forming a covalent bond. In some embodiments, the change is by changing the composition (chemical change such as protonation/de-protonation), changing its electronic structure (oxidation/reduction) of the detectable label.

The term “compound” in the context of detecting one or more types of cell, bacteria or bacteriophage refers to a molecule or a group of molecules that interacts a nucleic acid or a protein of one or more types of bacteriophage or bacteria, or bacterial cell wall or cell membrane. In some embodiments, the compound is permeable to bacterial cell wall or cell membrane. In some embodiments, the compound binds to dsDNA, ssDNA, dsRNA, or ssRNA. In some embodiments, the compound preferentially binds to dsDNA. In some embodiments, the compound is photoreactive. Non-limiting examples of compound binding to a nucleic acid include propidium monoazide, ethidium monoazide, based dye, propidium iodide, ethidium bromide, phenanthridine dye, acridine dye, indoles, or imidazole dye.

In some embodiments, compound binds covalently to a nucleic acid or a protein. In some embodiments, the compound intercalates between the bases of a nucleic acid. In some embodiments, the compound is modified upon contacting a bacteriophage or bacterial nucleic acid or protein. Non-limiting examples of methods of modification include forming a covalent bond, changing its composition, and changing its electronic structure. In some embodiments, the compound binds to a function group of a protein such as amine, carboxyl, phosphate, aldehyde, thiol, hydroxyl, and carbonyl. In some embodiments, the compound binding to a protein is an antibody. In some embodiments, the compound binding to a protein may be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, and alkyne.

In some embodiments, the compound has a low dissociation constant. In some embodiments, the compound upon binding to a protein or a nucleic acid prevents other type of compounds binding to the same protein or nucleic acid. In some embodiments, a compound preferentially binds to a protein or nucleic acid in the presence of another protein or nucleic acid which is bound to a different compound. In some embodiments, this preferential binding of a compound to one set of nucleic acid or protein in the presence of another set of nucleic acid or protein that is bound to a different compound is at least in part because the different compound prevents binding of the compound to the another set of nucleic acid or protein.

In some embodiments, the compound comprises a detectable label.

Solid Support

Solid support can be two- or three-dimensional and can comprise a planar surface (e.g., a glass slide) or can be shaped. A solid support can include glass (e.g., controlled pore glass (CPG)), corrugated glass, quartz, plastic (such as polystyrene (low cross-linked and high cross-linked polystyrene), polycarbonate, polypropylene and poly(methylmethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional solid support include, for example, spheres, microparticles, beads, membranes, slides, plates, micromachined chips, tubes (e.g., capillary tubes), microwells, microfluidic devices, flow cells, channels, filters, fluidic cartridge. Solid support can include planar arrays or matrices capable of having regions that include populations of bacteriophage or bacteria.

In some embodiments, the solid support or its surface is non-planar, such as the inner or outer surface of a tube or vessel. In some embodiments, the solid support is a surface of a flow cell.

In some embodiments, solid support may include Surface Enhanced Raman Spectroscopy (SERS) surface. In some embodiments, solid support may include a base support and a metallic nanostructure layer deposited upon the base support. The nanomaterial employed in the nanostructure layer may be any SERS-active metallic material, such as silver, gold, copper, platinum, titanium, chromium, combinations thereof or the like. In addition, the nanostructure layer may assume any form, such as nanoparticles, nanoaggregates, nanopores/nanodisks, nanorods, nanowires, or combinations thereof. In some embodiments, the nanoparticles are arranged on the base support by self-assembly. The base support may be any ceramic, polymer, metal, silicon, quartz (crystalline silica), glass, zinc oxide, alumina, Paraffin film, polycarbonate (PC), combinations thereof and the like. In some embodiments, the solid support is hollow metal or metal oxide nano- or microspheres.

In some embodiments, the solid support comprises microspheres or beads. As used herein, “microspheres” or “beads” or “particles” or grammatical equivalents herein is meant to include small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon, as well as any other materials outlined herein for solid supports may all be used. “Microsphere Detection Guide” from Bangs Laboratories, Fishers Ind. is a helpful guide. In certain embodiments, the microspheres are magnetic microspheres or beads. In some embodiments, the beads can be color coded. For example, MicroPlex® Microspheres from Luminex, Austin, Tex. may be used.

The beads need not be spherical; irregular particles may be used. Alternatively or additionally, the beads may be porous. The bead sizes range from nanometers, i.e. 10 nm, to millimeters, i.e. 1 mm, with beads from about 0.2 micron to about 200 microns being preferred, and from about 0.5 to about 5 micron being particularly preferred, although in some embodiments smaller or larger beads may be used. In some embodiments, beads can be about 0.1, 0.2, 0.5, 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter.

In some embodiments, the solid support has at least one of the components (materials) transparent to electromagnetic radiation. In some embodiments, the transparent substrate is conductive. In some embodiments, the transparent substrate is dielectric. In some embodiments, the solid support has at least one of its components opaque to electromagnetic radiation. In some embodiments, the opaque substrate is conductive. In some embodiments, the opaque substrate is dielectric. In some embodiments, the conductive component (material) is porous. In some embodiments, the dielectric component is porous. In some embodiments, the pore size is less than 10 nm. In some embodiments, one of the components is magnetic. In some embodiments, the substrate is has a periodic structure. In some embodiments, the period of the substrate is less than a micron in dimension. In some embodiments, the period of the substrate is more than a micron in dimension. In some embodiments, the substrate has a combination of the above embodiments. In some embodiments, the substrates mentioned above generate an electromagnetic resonance.

In some embodiments, the solid support is a sensor. In some embodiments, the sensor is a piezoelectric sensor. In some embodiments, the piezoelectric sensor has at least a monolayer of gold on top or bottom of the solid support. In some embodiments, the sensors are round-shaped. In some embodiments, solid support comprises quartz crystals with gold coating. In some embodiments, the sensors are mounted on a flowcell.

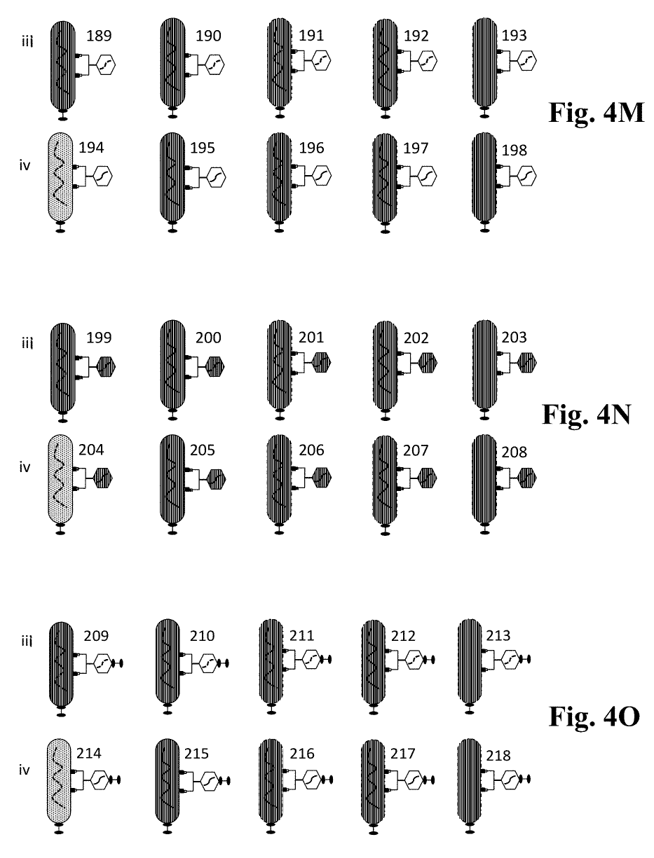

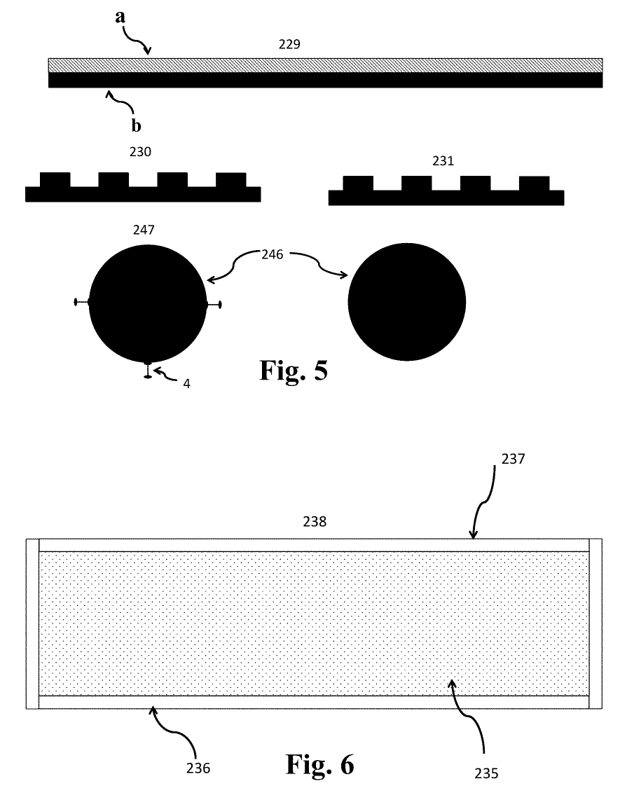

FIG. 5 shows exemplary solid supports. In some embodiments, the solid support comprises plurality of materials of defined compositions and property. In some embodiments, the solid support comprises a solid surface 229 with bilayer “a” and “b” of different chemical composition. In some embodiments, one layer may be conductive of electricity. In some embodiments, one layer may be a metal. Exemplary metals include, but are not limited to silver, gold, copper, platinum, titanium, chromium, Al, Ni, Cr, Fe, ceramic metals such as Indium-tin-oxide etc. or combinations thereof. In some embodiments, one layer comprises magnetic materials, e.g., Fe, Ni, Non-oxide, non-elemental magnetic material Nd2Fe14B, SmCo5 etc., non-elemental, oxide magnetic material Fe₃O₄, Fe₂O₃. In some embodiments, the second layer can be non-conductive or non-magnetic. In some embodiments, the second layer may be ceramic, polymer, metal, silicon, quartz (crystalline silica), glass, zinc oxide, alumina, Paraffin film, polycarbonate (PC), or combinations thereof.

In some embodiments, the solid support is a microarray 230 or 231. In some embodiments, the mircroarray 231 may comprise different materials for the wells and the base. In some embodiments, the solid support is a bead 246 and 247. In some embodiments, the bead 247 may comprise detectable label 4.

In some embodiments, the bacteria, bacteriophage, and/or the bacteria and bacteriophage complex may be immobilized on a solid support. In some embodiments, bacteria are immobilized on a solid support. In some embodiments, bacteriophage is immobilized on a solid support. In some embodiments, bacteria and bacteriophage complex is immobilized on a solid support. In some embodiments, the bacteria, bacteriophage, or the bacteria and bacteriophage complex are adsorbed passively on the substrate. In some embodiments, at least one bacterium, one bacteriophage, or one bacteria and bacteriophage complex is actively adsorbed by applying an external electric field. In some embodiments, the immobilization is performed under with buffers of varying concentrations and pH.

In some embodiments, the immobilization of the bacteria, bacteriophage, and/or the bacteria and bacteriophage complex to the solid support can be achieved through direct or indirect bonding to the solid support. In some embodiments, the bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. In some embodiments, the solid support comprises functional groups capable of immobilizing the bacteria, bacteriophage, or the bacteria and bacteriophage complex.

In some embodiments, the solid supports may comprise functional groups capable of covalently linking the bacteria, bacteriophage, or the bacteria and bacteriophage complex directly or indirectly through chemical linkers. Examples of functional groups include but are not limited to poly L-lysine, aminosilane, epoxysilane, aldehydes, carboxylic groups, azide, alkalyne, maleimide, amino groups, epoxy groups, cyano groups, ethylenic groups, hydroxyl groups, thiol groups.

In some embodiments, the immobilization is achieved through amine functional groups of a polymer, e.g., N-terminus and ε-amino groups of lysine residues, direct amine bonding of a terminal nucleotide of the template or a primer. In some embodiments, the immobilization is achieved through carboxylic acid/carboxylate functional groups of a polymer, e.g., C-terminus of a protein or a peptide. In some embodiments, the polymer is an aptamer.

In some embodiments, the solid support comprises epoxide functional groups, N-Hydroxysuccinimide (NHS) group. The epoxide functional groups or the NHS group can be used to immobilize the bacteria, bacteriophage, or the bacteria and bacteriophage complex to the solid support.

In some embodiments, the immobilization of the bacteria, bacteriophage, and/or the bacteria and bacteriophage complex to the solid support can be achieved through non-covalent means. In some embodiments, non-covalently immobilizing the bacteria, bacteriophage, and/or the bacteria and bacteriophage complex to the solid support is via a “binding pair,” which refers herein to two molecules which form a complex through a specific interaction. Thus, the bacteria, bacteriophage, and/or the bacteria and bacteriophage complex can be immobilized on the solid support through an interaction between one member of the binding pair linked to the bacteria, bacteriophage, and/or the bacteria and bacteriophage complex and the other member of the binding pair coupled to the solid support. In a preferred embodiment, the binding pair is biotin and avidin, or variants of avidin such as streptavidin or NeutrAvidin™. In some embodiments, the solid support may comprise streptavidin or its variants and bacteria, bacteriophage, and/or the bacteria and bacteriophage complex may comprise biotin. Methods for biotinylating are known in the art (e.g. through primary amine by NHS-PEO12-Biotin, NHS-LC-LC-Biotin, NHS-SS-PEO4-Biotin from Pierce Chemical Co.; through sulfhydryl group by Maleimide-PEO11-Biotin, Biotin-BMCC Sulfhydryl, Iodacetyl-PEO2-Biotin).

In other embodiments, the binding pair consists of a ligand-receptor, a hormone-receptor, an antigen-antibody. Examples of such binding pair include but are not limited to digoxigenin and anti-digoxigenin antibody; 6-(2,4-dinitrophenyl) aminohexanoic acid and anti-dinitrophenyl antibody; 5-Bromo-dUTP (BrdUTP) and anti-BrdUTP antibody; N-acetyl 2-aminofluorene (AAF) and anti-AAF antibody.

The terms “detectable label” and “tag” have been used interchangeably throughout the application and refers to a molecule or a compound or a group of molecules or a group of compounds and being capable of being detected. In some embodiments, detectable label can associate with a molecule and assist in detecting the molecule with which it is associated with.

In some embodiments, detectable label can generate a signal which can be detected. In some embodiments, the detectable label is converted to a different molecule or compound that can generate a signal which can be detected. In some embodiments, detectable label is part of a binding pair. In some embodiments, the detectable label is associated with a solid support. In some embodiments, the detectable label is associated with a bead. In some embodiments, the detectable is further modified prior to detection. In some embodiments, such modification may include covalent modification. In some embodiments, the detectable label is associated with the bacteriophage, and/or the bacteria prior to the formation of the complex of the bacteriophage and the bacteria. In some embodiments, the detectable label is associated with the bacteriophage, bacteria, and/or the complex of bacteriophage and the bacteria after the formation of the complex.

In some embodiments, the detectable label is associated with a protein or a nucleic acid (e.g., genomic nucleic acid, fragments of genomic nucleic acid, a probe or primer) of bacteria, bacteriophage, or a complex of bacteria and bacteriophage and is used to detect the bacteria, bacteriophage, or a complex of bacteria and bacteriophage. In some embodiments, the detectable label is associated with a protein or a nucleic acid inside the bacteria, bacteriophage, or a complex of bacteria and bacteriophage.

In some embodiments, the bacteriophage, bacteria, and/or the complex of bacteriophage and the bacteria may comprise more than one type of detectable label. In some embodiments, the detectable labels of the bacteriophage, the bacteria, and their complexes are different from each other.

In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluoresence, or chemiluminescence, or any other appropriate means.

Detectable labels include but are not limited to fluorophores, isotopes (e.g., 32P, 33P, 35S, 31I, 14C, 125I, 131I), electron-dense reagents (e.g., gold, silver), nanoparticles, enzymes commonly used in an ELISA (e.g., horseradish peroxidase, beta-galactosidase, luciferase, alkaline phosphatase), chemiluminiscent compound, colorimetric labels (e.g., colloidal gold), magnetic labels (e.g. Dynabeads™), biotin, digoxigenin, haptens, proteins for which antisera or monoclonal antibodies are available, ligands, hormones, oligonucleotides capable of forming a complex with the corresponding oligonucleotide complement.

In some embodiments, the detectable label comprises a fluorescent moiety. Non-limiting examples of fluorescent moiety include BOBO™, POPO™, SYBR®, SYTOX®, Cy2, Cy3, Cy3.5, Cy5, Cy5.5, Cy7, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED™, SPECTRUM GREEN, cyanine dyes (e.g., CY-3™, CY-5™, CY-3.5™, CY-5.5™, etc.), Alexa Fluor® dyes (e.g., Alexa Fluor® 350, Alexa Fluor® 405, Alexa Fluor® 430, Alexa Fluor® 488, Alexa Fluor® 532, Alexa Fluor® 546, Alexa Fluor® 568, Alexa Fluor® 594, Alexa Fluor(632, Alexa Fluor® 633, Alexa Fluor® 660, Alexa Fluor® 680, etc.), BODIPY™dyes (e.g., BODIPY™ FL, BODIPY™ R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™630/650, BODIPY™650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable label may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92: 4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described previously, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

A suitable detectable label can be an intercalating DNA/RNA dye that have dramatic fluorescent enhancement upon binding to double-stranded DNA/RNA. Examples of suitable dyes include, but are not limited to, SYBR™ and Pico Green (from Molecular Probes, Inc. of Eugene, Oreg.), ethidium bromide, propidium iodide, chromomycin, acridine orange, Hoechst 33258, TOTO-1, YOYO-1, and DAPI (4′,6-diamidino-2-phenylindole hydrochloride). Additional discussion regarding the use of intercalation dyes is provided by Zhu et al., Anal. Chem. 66:1941-1948 (1994), which is incorporated by reference in its entirety.

As used herein, the term “bacteria” refers to both gram positive and gram-negative bacteria. In some embodiments, the bacteria are pathogenic to mammals. In some embodiments, the bacteria are non-pathogenic to mammals. In some embodiments, the bacteria are non-pathogenic to humans. In some embodiments, the bacteria are mesophilic. In some embodiments, the bacteria are thermophilic.

In some embodiments, the bacteria are used in dairy fermentation, malolactic fermentation in wine making, in meat fermentation, vegetable fermentation, food fermentation, vinegar fermentation. In some embodiments, the bacteria are susceptible to bacteriophage infection. In some embodiments, the following the phage infection, the bacterial cells are lysed. In some embodiments, the following the phage infection, the bacterial cells are not lysed.

As used herein the term “susceptible to the bacteriophage” refers to bacteria that have receptors for the bacteriophage that are present in a fermentation sample. As a result, the bacteriophages present in a fermentation sample can specifically bind to such receptors and infect the bacteria.

FIG. 2 depicts various forms of bacteria that are susceptible to the bacteriophage present in a fermentation sample. As shown in FIG. 2, in some embodiments, the nucleic acid 24 of the susceptible bacteria is not associated with a detectable label or in some embodiments, the nucleic acid 25 of the susceptible bacteria is associated with a detectable label. The susceptible bacteria comprise a receptor molecule 26 for the bacteriophage. The susceptible bacteria can be associated with optional second detectable label 4 and a third detectable label 5. In some embodiments, the detectable labels change the physical and/or chemical properties and compositions of the virulent bacteriophage. FIG. 2 shows various combinations of the detectable labels and the susceptible bacteria: 27—susceptible bacteria without any detectable label; 28—susceptible bacteria with detectable label associated with the nucleic acid 25; 29—susceptible bacteria without any detectable label in which the cell wall or the cell membrane or both are compromised; 30—susceptible bacteria with detectable label associated with the nucleic acid 25 and whose cell wall or the cell membrane or both are compromised; 31—a ghost cell without bacterial nucleic acid; 32—susceptible bacteria with a third detectable label 5 and nucleic acid 24; 33—susceptible bacteria with a third detectable label 5 and nucleic acid 25; 34—susceptible bacteria 29 with a third detectable label 5 and nucleic acid 24; 35—susceptible bacteria 30 with a third detectable label 5 and nucleic acid 25; 36—ghost cell with a third detectable label 5; 37—susceptible bacteria 27 with second detectable label 4; 38—susceptible bacteria 28 with second detectable label 4; 39—susceptible bacteria 29 with second detectable label 4; 40—susceptible bacteria 30 with second detectable label 4; 41—ghost cell 31 with second detectable label 4; 42—susceptible bacteria 27 with second and third detectable labels 4 and 5, respectively; 43—susceptible bacteria 28 with second and third detectable labels 4 and 5, respectively; 44—susceptible bacteria 29 with second and third detectable labels 4 and 5, respectively; 45—susceptible bacteria 30 with second and third detectable labels 4 and 5, respectively; 46—ghost cell 31 with second and third detectable labels 4 and 5, respectively.

FIG. 3 depicts various forms of bacteria that are not susceptible to the bacteriophage present in a fermentation sample. In some embodiments, the bacteria are used in dairy fermentation. In some embodiments, the bacteria are used in malolactic acid fermentation to convert malic acid to lactic acid in wine industry. As shown in FIG. 3, in some embodiments, the nucleic acid 47 of the non-susceptible bacteria is not associated with a detectable label or in some embodiments, the nucleic acid 48 of the non-susceptible bacteria is associated with a detectable label. The susceptible bacteria can be associated with optional second detectable label 4 and a third detectable label 5. In some embodiments, the detectable labels change the physical and/or chemical properties and compositions of the virulent bacteriophage. FIG. 3 shows various combinations of the detectable labels and the non-susceptible bacteria: 49—non-susceptible bacteria without any detectable label; 50—non-susceptible bacteria with detectable label associated with the nucleic acid 48; 51—non-susceptible bacteria without any detectable label in which the cell wall or the cell membrane or both are compromised; 52—non-susceptible bacteria with detectable label associated with the nucleic acid 48 and whose cell wall or the cell membrane or both are compromised; 53—a ghost cell without bacterial nucleic acid; 54—non-susceptible bacteria 49 with a third detectable label 5 and nucleic acid 47; 55—non-susceptible bacteria 50 with a third detectable label 5 and nucleic acid 48; 56—non-susceptible bacteria 51 with a third detectable label 5 and nucleic acid 47; 57—non-susceptible bacteria 52 with a third detectable label 5 and nucleic acid 48; 58—ghost cell with a third detectable label 5; 59—non-susceptible bacteria 49 with second detectable label 4; 60—non-susceptible bacteria 50 with second detectable label 4; 61—non-susceptible bacteria 51 with second detectable label 4; 62—non-susceptible bacteria 52 with second detectable label 4; 63—ghost cell 53 with second detectable label 4; 64—non-susceptible bacteria 49 with second and third detectable labels 4 and 5, respectively; 65—non-susceptible bacteria 50 with second and third detectable labels 4 and 5, respectively; 66—non-susceptible bacteria 51 with second and third detectable labels 4 and 5, respectively; 67—non-susceptible bacteria 52 with second and third detectable labels 4 and 5, respectively; 68—ghost cell 53 with second and third detectable labels 4 and 5, respectively.

As used herein, the term “bacteriophage” refers to a virus that infects and can replicate within a bacterium. Bacteriophages may have a lytic cycle, a lysogenic cycle, or both. Bacteriophages attach to specific receptors on the surface of bacteria, including lipopolysaccharides, teichoic acids, proteins, or even flagella to form a bacteria and bacteriophage complex. This specificity means a bacteriophage can infect only certain bacteria bearing receptors to which they can bind, which in turn determines the phage's host range. Host growth conditions also influence the ability of the phage to attach and invade them. Genomes of the bacteriophages can be RNA or DNA. In some embodiments, the genome of the bacteriophage can be linear dsDNA, circular dsDNA, circular ssDNA, segmented dsRNA, linear ssRNA. The classification by the International Committee on Taxonomy of Viruses (ICTV) according to morphology and nucleic acid is shown in Table 1.

TABLE 1
ICTV classification of prokaryotic (bacterial and archaeal) viruses

| | | | Nucleic | |
|---|---|---|---|---|
| Order | Family | Morphology | acid | Examples |
| Caudovirales | Myoviridae | Nonenveloped, | Linear | T4 phage, Mu, PBSX, |
| | | contractile tail | dsDNA | P1Puna-like, P2, I3, |
| | | | | Bcep 1, Bcep 43, |
| | | | | Bcep 78 |
| Caudovirales | Siphoviridae | Nonenveloped, | Linear | λ phage, T5 phage, |
| | | noncontractile tail | dsDNA | phi, C2, L5, HK97, |
| | | (long) | | N15 |
| Caudovirales | Podoviridae | Nonenveloped, | Linear | T7 phage, T3 |
| | | noncontractile tail | dsDNA | phage, Φ29, P22, P37 |
| | | (short) | | |
| Ligamenvirales | Lipothrixviridae | Enveloped, rod- | Linear | Acidianus |
| | | shaped | dsDNA | filamentous virus 1 |
| Ligamenvirales | Rudiviridae | Nonenveloped, | Linear | Sulfolobus islandicus |
| | | rod-shaped | dsDNA | rod-shaped virus 1 |
| Unassigned | Ampullaviridae | Enveloped, bottle- | Linear | |
| | | shaped | dsDNA | |
| Unassigned | Bicaudaviridae | Nonenveloped, | Circular | |
| | | lemon-shaped | dsDNA | |
| Unassigned | Clavaviridae | Nonenveloped, | Circular | |
| | | rod-shaped | dsDNA | |
| Unassigned | Corticoviridae | Nonenveloped, | Circular | |
| | | isometric | dsDNA | |
| Unassigned | Cystoviridae | Enveloped, | Segmented | |
| | | spherical | dsRNA | |
| Unassigned | Fuselloviridae | Nonenveloped, | Circular | |
| | | lemon-shaped | dsDNA | |
| Unassigned | Globuloviridae | Enveloped, | Linear | |
| | | isometric | dsDNA | |
| Unassigned | Guttaviridae | Nonenveloped, | Circular | |
| | | ovoid | dsDNA | |
| Unassigned | Inoviridae | Nonenveloped, | Circular | M13 |
| | | filamentous | ssDNA | |
| Unassigned | Leviviridae | Nonenveloped, | Linear | MS2, Qβ |
| | | isometric | ssRNA | |
| Unassigned | Microviridae | Nonenveloped, | Circular | ΦX174 |
| | | isometric | ssDNA | |
| Unassigned | Plasmaviridae | Enveloped, | Circular | |
| | | pleomorphic | dsDNA | |
| Unassigned | Tectiviridae | Nonenveloped, | Linear | |
| | | isometric | dsDNA | |

In some embodiments, the bacteriophages are lytic phages. In some embodiments, the bacteriophages belong to Siphoviridae and Podoviridae families. In some embodiments, the bacteriophages are responsible for milk fermentation failures. In some embodiments, the bacteriophages are responsible for infecting the bacteria useful for bacterial fermentation of milk. In some embodiments, the bacteriophages are responsible for infecting the bacteria useful for malolactic fermentation in wine industry.

Non-limiting examples of bacteria used for fermentation in dairy and wine industry and their corresponding bacteriophages are shown in Table 2 below.

TABLE 2
bacteria used for fermentation in dairy and wine industry
and their corresponding bacteriophages

| Bacteria | Phage |
|---|---|
| Lactobacillus casei | A2 |
| Lactobacillus paracasei | CL1, CL2 |
| Lactobacillus paracasei | PL-1 |
| (ssp. paracasei) | |
| Lactobacillus plantarum | B1, B2, PhiJL-1, fri, Φg1e, |
| | ΦLP65, ΦJL1 |
| Lactococcus lactis | P001, 949, c6A, P087, P107, |
| | BK5-T, c2, p2, sk1, ul36, bIL170, |
| | bIL67, Q54, 1706, P335, |
| | Tuc2009, r1t |
| Lactococcus lactis (ssp. cremoris) | 1358, 936, KSY1 |
| Lactococcus lactis (ssp. diacetylactis) | P008, P270, P369 |
| Lactococcus lactis (ssp. lactis) | 1483 |
| Lactobacillus casei | A2, ΦAT3, ΦA2, |
| Lactobacillus paracasei | CL1, CL2 |
| Lactobacillus paracasei | PL-1 |
| (ssp. paracasei) | |
| Oenococcus oeni | Lco22 |
| Streptococcus group C | a/C7 |
| Streptococcus mitis | |
| Streptococcus mutans | M102, M102AD |
| Streptococcus pneumoniae | Cp-1 (SOCP), Dp-1 |
| Streptococcus thermophilus | Ba 24, DT1, Q1, 2972, 858, |
| | ALQ13.2, ΦAbc2, |
| Thermus thermophilus | ψYS40 |
| Leuconostoc mesenteroides | pro2 |
| Leuconostoc mesenteroides | Φ400 |
| (ssp. cremoris) | |
| Lactobacillus delbrueckii | LdlS, Ld3, Ld17, Ld25A, lb539, |
| | LL-Ku, c5, JCL1032, ΦLL-H, |
| | Φmv4, |
| Lactobacillus helveticus | ΦAQ113 |
| Lactobacillus gasseri | KC5a |
| Lactobacillus johnsonii | Lj771 |
| Lactobacillus gasseri | Φadh, ΦKC5a |
| Lactobacillus rhamnosus | ΦLc-Nu |
| Lactobacillus fermentum | EcoSau, EcoInf |

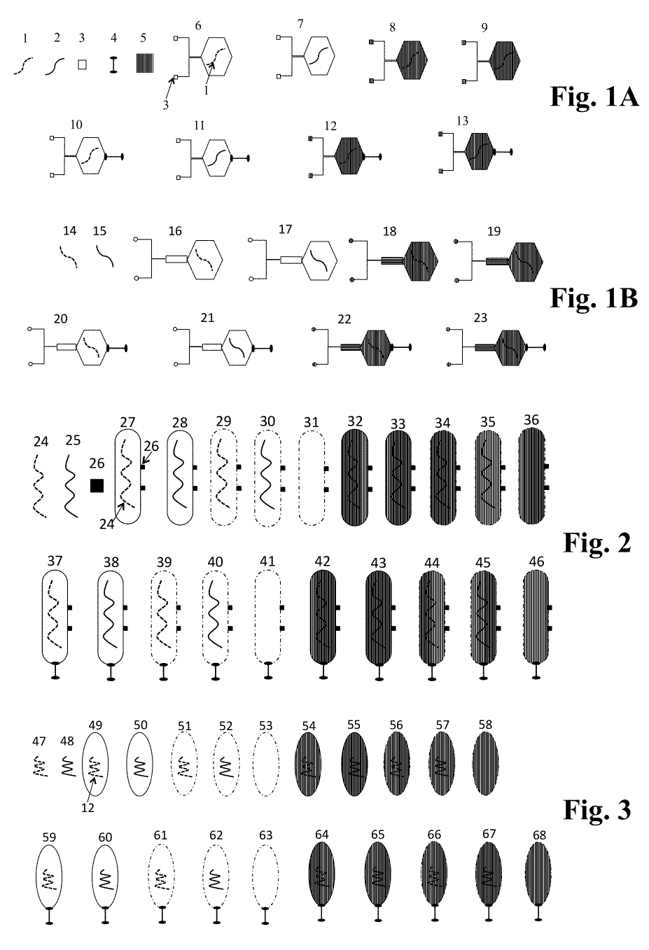

FIG. 1A depicts various forms of virulent bacteriophage that can infect the bacteria in a sample. As shown in FIG. 1A, in some embodiments, the nucleic acid 1 of the virulent bacteriophage is not associated with a detectable label or in some embodiments, the nucleic acid 2 of the virulent bacteriophage is associated with a detectable label. The virulent bacteriophage comprises a bacteria binding molecule 3. The bacteriophages can be associated with optional second detectable label 4 and a third detectable label 5. In some embodiments, the detectable labels change the physical and/or chemical properties and compositions of the virulent bacteriophage. FIG. 1A shows various combinations of the detectable labels and the bacteriophage: 6—virulent bacteriophage without any detectable label; 7—virulent bacteriophage with detectable label associated with the nucleic acid; 8—virulent bacteriophage with a second detectable label 4 and nucleic acid 1; 9—virulent bacteriophage with a second detectable label 4 and nucleic acid 2; 10—virulent bacteriophage with a third detectable label 5 and nucleic acid 1; 11—virulent bacteriophage with a third detectable label 5 and nucleic acid 2; 12—virulent bacteriophage with a second and third detectable labels 4 and 5, respectively and nucleic acid 1; 13—virulent bacteriophage with a second and third detectable labels 4 and 5, respectively and nucleic acid 2.

FIG. 1B depicts various forms of non-virulent bacteriophage that cannot infect the bacteria in a sample. As shown in FIG. 1B, in some embodiments, the nucleic acid 14 of the non-virulent bacteriophage is not associated with a detectable label or in some embodiments, the nucleic acid 15 of the non-virulent bacteriophage is associated with a detectable label. The bacteriophages can be associated with optional second detectable label 4 and a third detectable label 5. In some embodiments, the detectable labels change the physical and/or chemical properties and compositions of the non-virulent bacteriophage. FIG. 1B shows various combinations of the detectable labels and the bacteriophage: 16—non-virulent bacteriophage without any detectable label; 17—non-virulent bacteriophage with detectable label associated with the nucleic acid 15; 18—non-virulent bacteriophage with a second detectable label 4 and nucleic acid 14; 19—non-virulent bacteriophage with a second detectable label 4 and nucleic acid 15; 20—non-virulent bacteriophage with a third detectable label 5 and nucleic acid 14; 21—non-virulent bacteriophage with a third detectable label 5 and nucleic acid 15; 22—non-virulent bacteriophage with a second and third detectable labels 4 and 5, respectively and nucleic acid 14; 23—non-virulent bacteriophage with a second and third detectable labels 4 and 5, respectively and nucleic acid 15.

As used herein the phrase “complex of bacteriophage and bacteria” or the “complex” refers to a state in the bacteriophage infection in which the bacteriophages attach to specific receptors on the surface of bacteria, including lipopolysaccharides, teichoic acids, proteins, or even flagella to form a bacteria and bacteriophage complex. The complex includes stages prior to, during, or after introduction of bacteriophage genetic material into the bacteria while the bacteriophage remains adhered to the bacterial surface.

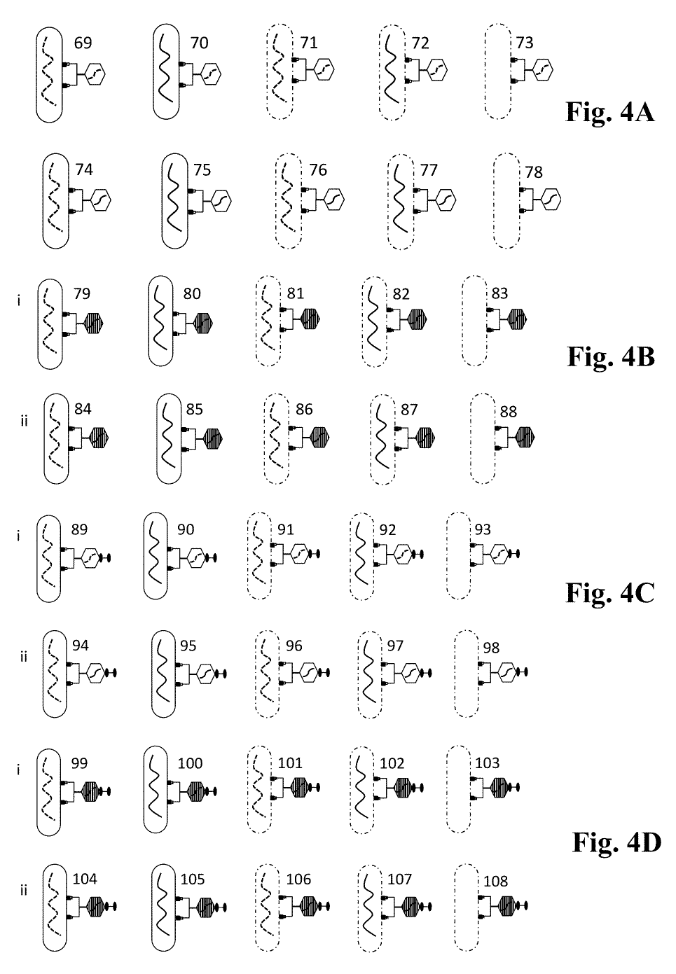

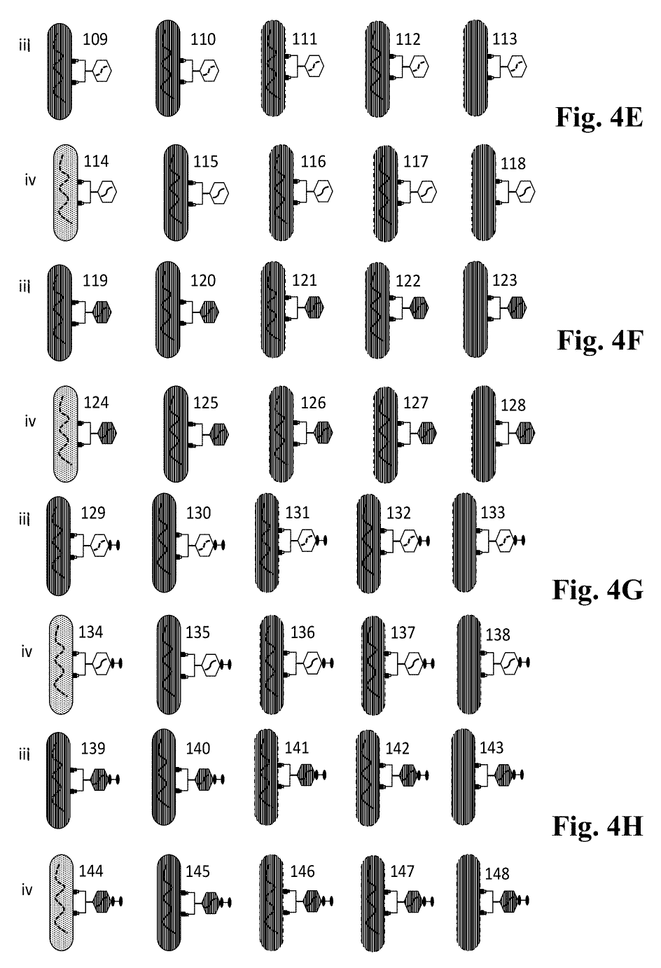

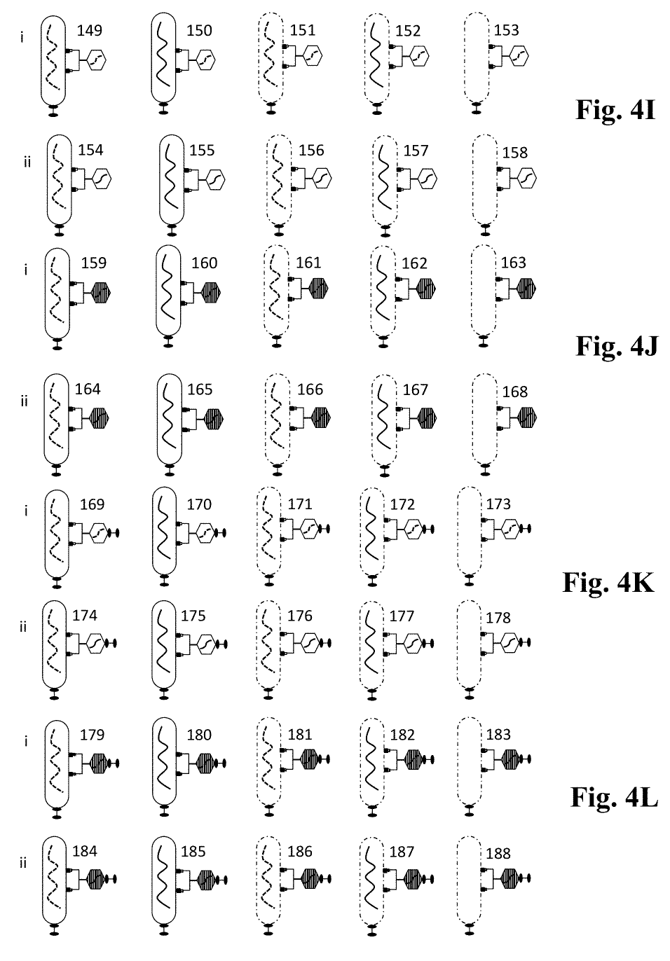

FIGS. 4 A-P depict various forms of complexes formed between the bacteriophage and the bacteria. Complex 69 is formed between virulent bacteriophage 6 and susceptible bacteria 27. Complex 70 is formed between virulent bacteriophage 6 and susceptible bacteria 28. Complex 71 is formed between virulent bacteriophage 6 and susceptible bacteria 29. Complex 72 is formed between virulent bacteriophage 6 and susceptible bacteria 30. Complex 73 is formed between virulent bacteriophage 6 and ghost cell 31.

Complex 74 is formed between virulent bacteriophage 7 and susceptible bacteria 27. Complex 75 is formed between virulent bacteriophage 7 and susceptible bacteria 28. Complex 76 is formed between virulent bacteriophage 7 and susceptible bacteria 29. Complex 77 is formed between virulent bacteriophage 7 and susceptible bacteria 30. Complex 78 is formed between virulent bacteriophage 7 and ghost cell 31.

Complex 79 is formed between virulent bacteriophage 8 and susceptible bacteria 27. Complex 80 is formed between virulent bacteriophage 8 and susceptible bacteria 28. Complex 81 is formed between virulent bacteriophage 8 and susceptible bacteria 29. Complex 82 is formed between virulent bacteriophage 8 and susceptible bacteria 30. Complex 83 is formed between virulent bacteriophage 8 and ghost cell 31.

Complex 84 is formed between virulent bacteriophage 9 and susceptible bacteria 27. Complex 85 is formed between virulent bacteriophage 9 and susceptible bacteria 28. Complex 86 is formed between virulent bacteriophage 9 and susceptible bacteria 29. Complex 87 is formed between virulent bacteriophage 9 and susceptible bacteria 30. Complex 88 is formed between virulent bacteriophage 9 and ghost cell 31.

Complex 89 is formed between virulent bacteriophage 10 and susceptible bacteria 27. Complex 90 is formed between virulent bacteriophage 10 and susceptible bacteria 28. Complex 91 is formed between virulent bacteriophage 10 and susceptible bacteria 29. Complex 92 is formed between virulent bacteriophage 10 and susceptible bacteria 30. Complex 93 is formed between virulent bacteriophage 10 and ghost cell 31.

Complex 94 is formed between virulent bacteriophage 11 and susceptible bacteria 27. Complex 95 is formed between virulent bacteriophage 11 and susceptible bacteria 28. Complex 96 is formed between virulent bacteriophage 11 and susceptible bacteria 29. Complex 97 is formed between virulent bacteriophage 11 and susceptible bacteria 30. Complex 98 is formed between virulent bacteriophage 11 and ghost cell 31.

Complex 99 is formed between virulent bacteriophage 12 and susceptible bacteria 27. Complex 100 is formed between virulent bacteriophage 12 and susceptible bacteria 28. Complex 101 is formed between virulent bacteriophage 12 and susceptible bacteria 29. Complex 102 is formed between virulent bacteriophage 12 and susceptible bacteria 30. Complex 103 is formed between virulent bacteriophage 12 and ghost cell 31.

Complex 104 is formed between virulent bacteriophage 13 and susceptible bacteria 27. Complex 105 is formed between virulent bacteriophage 13 and susceptible bacteria 28. Complex 106 is formed between virulent bacteriophage 13 and susceptible bacteria 29. Complex 107 is formed between virulent bacteriophage 13 and susceptible bacteria 30. Complex 108 is formed between virulent bacteriophage 12 and ghost cell 31.

Complex 109 is formed between virulent bacteriophage 6 and susceptible bacteria 32. Complex 110 is formed between virulent bacteriophage 6 and susceptible bacteria 33. Complex 111 is formed between virulent bacteriophage 6 and susceptible bacteria 34. Complex 112 is formed between virulent bacteriophage 6 and susceptible bacteria 35. Complex 113 is formed between virulent bacteriophage 6 and ghost cell 36.

Complex 114 is formed between virulent bacteriophage 7 and susceptible bacteria 32. Complex 115 is formed between virulent bacteriophage 7 and susceptible bacteria 33. Complex 116 is formed between virulent bacteriophage 7 and susceptible bacteria 34. Complex 117 is formed between virulent bacteriophage 7 and susceptible bacteria 35. Complex 118 is formed between virulent bacteriophage 6 and ghost cell 36.

Complex 119 is formed between virulent bacteriophage 8 and susceptible bacteria 32. Complex 120 is formed between virulent bacteriophage 8 and susceptible bacteria 33. Complex 121 is formed between virulent bacteriophage 8 and susceptible bacteria 34. Complex 122 is formed between virulent bacteriophage 8 and susceptible bacteria 35. Complex 123 is formed between virulent bacteriophage 8 and ghost cell 36.

Complex 124 is formed between virulent bacteriophage 9 and susceptible bacteria 32. Complex 125 is formed between virulent bacteriophage 9 and susceptible bacteria 33. Complex 126 is formed between virulent bacteriophage 9 and susceptible bacteria 34. Complex 127 is formed between virulent bacteriophage 9 and susceptible bacteria 35. Complex 128 is formed between virulent bacteriophage 9 and ghost cell 36.

Complex 129 is formed between virulent bacteriophage 10 and susceptible bacteria 32. Complex 130 is formed between virulent bacteriophage 10 and susceptible bacteria 33. Complex 131 is formed between virulent bacteriophage 10 and susceptible bacteria 34. Complex 132 is formed between virulent bacteriophage 10 and susceptible bacteria 35. Complex 133 is formed between virulent bacteriophage 10 and ghost cell 36.

Complex 134 is formed between virulent bacteriophage 11 and susceptible bacteria 32. Complex 135 is formed between virulent bacteriophage 11 and susceptible bacteria 33. Complex 136 is formed between virulent bacteriophage 11 and susceptible bacteria 34. Complex 137 is formed between virulent bacteriophage 11 and susceptible bacteria 35. Complex 138 is formed between virulent bacteriophage 11 and ghost cell 36.

Complex 139 is formed between virulent bacteriophage 12 and susceptible bacteria 32. Complex 140 is formed between virulent bacteriophage 12 and susceptible bacteria 33. Complex 141 is formed between virulent bacteriophage 12 and susceptible bacteria 34. Complex 142 is formed between virulent bacteriophage 12 and susceptible bacteria 35. Complex 143 is formed between virulent bacteriophage 12 and ghost cell 36.

Complex 144 is formed between virulent bacteriophage 13 and susceptible bacteria 32. Complex 145 is formed between virulent bacteriophage 13 and susceptible bacteria 33. Complex 146 is formed between virulent bacteriophage 13 and susceptible bacteria 34. Complex 147 is formed between virulent bacteriophage 13 and susceptible bacteria 35. Complex 148 is formed between virulent bacteriophage 13 and ghost cell 36.

Complex 149 is formed between virulent bacteriophage 6 and susceptible bacteria 37. Complex 150 is formed between virulent bacteriophage 6 and susceptible bacteria 38. Complex 151 is formed between virulent bacteriophage 6 and susceptible bacteria 39. Complex 152 is formed between virulent bacteriophage 6 and susceptible bacteria 40. Complex 153 is formed between virulent bacteriophage 6 and ghost cell 41.

Complex 154 is formed between virulent bacteriophage 7 and susceptible bacteria 37. Complex 155 is formed between virulent bacteriophage 7 and susceptible bacteria 38. Complex 156 is formed between virulent bacteriophage 7 and susceptible bacteria 39. Complex 158 is formed between virulent bacteriophage 7 and susceptible bacteria 40. Complex 158 is formed between virulent bacteriophage 7 and ghost cell 41.

Complex 159 is formed between virulent bacteriophage 8 and susceptible bacteria 37. Complex 160 is formed between virulent bacteriophage 8 and susceptible bacteria 38. Complex 161 is formed between virulent bacteriophage 8 and susceptible bacteria 39. Complex 162 is formed between virulent bacteriophage 8 and susceptible bacteria 40. Complex 163 is formed between virulent bacteriophage 8 and ghost cell 41.

Complex 164 is formed between virulent bacteriophage 9 and susceptible bacteria 37. Complex 165 is formed between virulent bacteriophage 9 and susceptible bacteria 38. Complex 166 is formed between virulent bacteriophage 9 and susceptible bacteria 39. Complex 167 is formed between virulent bacteriophage 9 and susceptible bacteria 40. Complex 168 is formed between virulent bacteriophage 9 and ghost cell 41.

Complex 169 is formed between virulent bacteriophage 10 and susceptible bacteria 37. Complex 170 is formed between virulent bacteriophage 10 and susceptible bacteria 38. Complex 171 is formed between virulent bacteriophage 10 and susceptible bacteria 39. Complex 172 is formed between virulent bacteriophage 10 and susceptible bacteria 40. Complex 173 is formed between virulent bacteriophage 10 and ghost cell 41.

Complex 174 is formed between virulent bacteriophage 11 and susceptible bacteria 37. Complex 175 is formed between virulent bacteriophage 11 and susceptible bacteria 38. Complex 176 is formed between virulent bacteriophage 11 and susceptible bacteria 39. Complex 177 is formed between virulent bacteriophage 11 and susceptible bacteria 40. Complex 178 is formed between virulent bacteriophage 11 and ghost cell 41.

Complex 179 is formed between virulent bacteriophage 12 and susceptible bacteria 37. Complex 180 is formed between virulent bacteriophage 12 and susceptible bacteria 38. Complex 181 is formed between virulent bacteriophage 12 and susceptible bacteria 39. Complex 182 is formed between virulent bacteriophage 12 and susceptible bacteria 40. Complex 183 is formed between virulent bacteriophage 12 and ghost cell 41.

Complex 184 is formed between virulent bacteriophage 13 and susceptible bacteria 37. Complex 185 is formed between virulent bacteriophage 13 and susceptible bacteria 38. Complex 186 is formed between virulent bacteriophage 13 and susceptible bacteria 39. Complex 187 is formed between virulent bacteriophage 13 and susceptible bacteria 40. Complex 188 is formed between virulent bacteriophage 13 and ghost cell 41.

Complex 189 is formed between virulent bacteriophage 6 and susceptible bacteria 42. Complex 190 is formed between virulent bacteriophage 6 and susceptible bacteria 43. Complex 191 is formed between virulent bacteriophage 6 and susceptible bacteria 44. Complex 192 is formed between virulent bacteriophage 6 and susceptible bacteria 45. Complex 193 is formed between virulent bacteriophage 6 and ghost cell 46.

Complex 194 is formed between virulent bacteriophage 7 and susceptible bacteria 42. Complex 195 is formed between virulent bacteriophage 7 and susceptible bacteria 43. Complex 196 is formed between virulent bacteriophage 7 and susceptible bacteria 44. Complex 197 is formed between virulent bacteriophage 7 and susceptible bacteria 45. Complex 198 is formed between virulent bacteriophage 7 and ghost cell 46.

Complex 199 is formed between virulent bacteriophage 8 and susceptible bacteria 42. Complex 200 is formed between virulent bacteriophage 8 and susceptible bacteria 43. Complex 201 is formed between virulent bacteriophage 8 and susceptible bacteria 44. Complex 202 is formed between virulent bacteriophage 8 and susceptible bacteria 45. Complex 203 is formed between virulent bacteriophage 8 and ghost cell 46.

Complex 204 is formed between virulent bacteriophage 9 and susceptible bacteria 42. Complex 205 is formed between virulent bacteriophage 9 and susceptible bacteria 43. Complex 206 is formed between virulent bacteriophage 9 and susceptible bacteria 44. Complex 207 is formed between virulent bacteriophage 9 and susceptible bacteria 45. Complex 208 is formed between virulent bacteriophage 9 and ghost cell 46.

Complex 209 is formed between virulent bacteriophage 10 and susceptible bacteria 42. Complex 210 is formed between virulent bacteriophage 10 and susceptible bacteria 43. Complex 211 is formed between virulent bacteriophage 10 and susceptible bacteria 44. Complex 212 is formed between virulent bacteriophage 10 and susceptible bacteria 45. Complex 213 is formed between virulent bacteriophage 10 and ghost cell 46.

Complex 214 is formed between virulent bacteriophage 11 and susceptible bacteria 42. Complex 215 is formed between virulent bacteriophage 11 and susceptible bacteria 43. Complex 216 is formed between virulent bacteriophage 11 and susceptible bacteria 44. Complex 217 is formed between virulent bacteriophage 11 and susceptible bacteria 45. Complex 218 is formed between virulent bacteriophage 11 and ghost cell 46.

Complex 219 is formed between virulent bacteriophage 12 and susceptible bacteria 42. Complex 220 is formed between virulent bacteriophage 12 and susceptible bacteria 43. Complex 221 is formed between virulent bacteriophage 12 and susceptible bacteria 44. Complex 222 is formed between virulent bacteriophage 12 and susceptible bacteria 45. Complex 223 is formed between virulent bacteriophage 12 and ghost cell 46.

Complex 224 is formed between virulent bacteriophage 13 and susceptible bacteria 42. Complex 225 is formed between virulent bacteriophage 13 and susceptible bacteria 43. Complex 226 is formed between virulent bacteriophage 13 and susceptible bacteria 44. Complex 227 is formed between virulent bacteriophage 13 and susceptible bacteria 45. Complex 228 is formed between virulent bacteriophage 13 and ghost cell 46.

In some embodiments, the complex is separated from the incubation mixture comprising bacteriophage and bacteria prior to detection of the complex. In some embodiments, the separation is by centrifugation, filtration, application of electric field, size exclusion chromatography or a combination thereof.

In some embodiments, the complex is detectably labeled after the formation of the complex. In some embodiments, the bacteriophage and/or the bacteria are detectably labeled resulting in a detectably labeled complex. In some embodiments, the detectable labels for the bacteriophage and the bacteria are different. In some embodiments, the detectable labels for the bacteriophage, the bacteria, and/or the complex are different. In some embodiments, the complex can comprise more than one type of detectable label.

As used herein the term “device” refers to a widget that aids in detecting or can detect bacteriophages or bacteriophage and bacteria complex in a sample.

In some embodiments, the device comprises a planar surface. In some embodiments, the planar surface comprises a bilayer 229 of two different compositions. In some embodiments, one layer may be conductive of electricity. In some embodiments, one layer may be a metal. Exemplary metals include, but are not limited to silver, gold, copper, platinum, titanium, chromium, Al, Ni, Cr, Fe, ceramic metals such as Indium-tin-oxide etc. or combinations thereof. In some embodiments, one layer comprises magnetic materials, e.g., Fe, Ni, Non-oxide, non-elemental magnetic material Nd₂Fe₁₄B, SmCo₅ etc., non-elemental, oxide magnetic material Fe₃O₄, Fe₂O₃. In some embodiments, the second layer can be non-conductive or non-magnetic. In some embodiments, the second layer may be ceramic, polymer, metal, silicon, quartz (crystalline silica), glass, zinc oxide, alumina, Paraffin film, polycarbonate (PC), or combinations thereof. In some embodiments, the planar surface is in fluid communication with the sample comprising bacteriophage, bacteria, and/or a complex of bacteriophage and bacteria.

In some embodiments, the device comprises solid support. In some embodiments, the solid supports may comprise functional groups capable of covalently linking the bacteriophage, bacteria, or the complex of bacteriophage and bacteria directly or indirectly through chemical linkers. Examples of functional groups include but are not limited to poly L-lysine, aminosilane, epoxysilane, aldehydes, amino groups, epoxy groups, cyano groups, ethylenic groups, hydroxyl groups, thiol groups, epoxide N-Hydroxysuccinimide (NHS) group. In some embodiments, the solid support may comprise one member of a binding pair and can immobilize a bacteriophage, bacteria, or the complex of bacteriophage and bacteria comprising the other member of the binding pair.

In some embodiments, the device may comprise a sensor. In some embodiments, the device may comprise plurality of sensors. In some embodiments, the sensor is a photo detector. In some embodiments, the photo detector is a spectrometer. In some embodiments, the sensor is a Raman spectrometer. In some embodiments, the sensor can detect surface plasmon resonance. In some embodiments, the sensor can detect change in mass. In some embodiments, the sensor can detect change in resonance frequency of the sensor. In some embodiments, the sensor comprises a crystal and the sensor can detect dissipation of shear movement of the crystal. In some embodiments, the sensor comprises piezoelectric crystal. In some embodiments, the sensor comprises piezoelectric crystal and a monolayer of a metal such as gold, silver etc. In some embodiments, the device comprises a sensor and a flow cell. In some embodiments, sample is flown over the sensor by a continuous flow.

In some embodiments, the device is fluidic device. In some embodiments, the fluidic device may comprise a solid support 236. In some embodiments, the solid support may be 229, 230, or 231. In some embodiments, fluid 235 is introduced to the solid support. The fluid may be stationary or the fluid may have a relative flow with respect to the solid support. In some embodiments, the fluid may comprise bacteriophage, bacteria, and/or complexes of bacteriophage and bacteria. In some embodiments, the fluidic device may have two solid supports 236 and 237 opposite to each other. In some embodiments, the fluidic device is a fluidic chamber 238. In some embodiments, bacteriophage, bacteria, and/or complexes of bacteriophage and bacteria are in contact with the solid support.

In some embodiments, the device comprises of at least one fluidic chamber or channel comprising at least two electrodes and at least one fluidic connection between the electrodes. In some embodiments, the device measures the membrane potential in which a change in the membrane potential is indicative of phage infection of a bacterial cell. In some embodiments, the device measures the redox potential of a molecule. In some embodiments, voltage sensitive dyes are used to measure response of the bacterial cells to phage infection. In some embodiments, application of an electric field between the electrodes allows the differential migration of the bacteriophage, bacteria, or the complex of the bacteriophage and the bacteria to the positive electrode. In some embodiments, the device may comprise a sensor to detect the differential migration of the bacteriophage, bacteria, or the complex of the bacteriophage and the bacteria. In some embodiments, a direct current is applied. In some embodiments, an alternate current is applied.

In some embodiments, the fluidic device comprises at least one main channel adapted to carry the fluid. In some embodiments, the channel comprises an interior wall. In some embodiments, the fluidic device comprises an inlet module upstream of the main channel and/or an outlet module downstream of the main channel. In some embodiments, the solid support comprises a hydrophobic surface. In some embodiments, the fluidic device comprises a force transducer that controls flow of the fluid. In some embodiments, the force transducer is an electric field, a magnetic field, a mechanical force, an optically induced force or any combination thereof.

In some embodiments, the device comprises a surface plasmon resonance (SPR) system. In some embodiments, the SPR system comprises a source of polarized light, a sensor comprising a transparent element such as a prism covered with a thin metal film and linking layer, a flow chamber, a system for controlling transport of fluid, and an optical unit for detecting reflected light. The SPR principle is based on the excitation of surface plasmons in a thin layer of a metal such as gold or silver, using polarized light. Polarized light from the prism is reflected from the metal surface, and at a certain angle of light incidence the excitation of resonance in the metal film results in intensity and phase changes in the reflected light beam. An evanescent field is generated which travels in a direction perpendicular to the surface, i.e. in the direction of the fluid sample. As a result of excitation of surface plasmons the incident light is adsorbed, resulting in a decrease in the intensity of the reflected light. For each wavelength and the corresponding effective refractive index ratios between both sides of the metal layer, there is a specific angle at which a minimum in reflectivity is observed, the SPR angle. This angle increases with decreasing wavelength. When interactions occur on the metal surface within the range of the penetration depth of the evanescent field while at the glass phase the refractive index remains constant, a change in refractive index of the dielectric fluid sample occurs. This change in refractive index causes a change in the angle at which SPR occurs. This change in SPR angle is recorded as a shift in the SPR angle with time, resulting in SPR sensorgrams. The change in SPR angle of reflected light is directly proportional to the binding of the bacteriophage, bacteria, and/or the complex of bacteriophage and bacteria on the metal surface of the prism.

In some embodiments, the device comprises a calorimeter. The device measures the heat of chemical reactions or physical changes, endothermic and exothermic peaks, enthalpy as well as heat capacity.

Detection

In some embodiments, the bacteriophage, bacteria, and/or the complex is excited by a specific electromagnetic radiation (excitation) with a defined energy and amplitude and the complex is detected by a specific electromagnetic spectral signature that is generated by the complex in response to the exciting electromagnetic radiation. In some embodiments, the complex is detected by spectrometer, optical microscope, Raman spectroscopy, surface enhanced Raman spectroscopy, SPR.

In some embodiments, the bacteriophages are detected by Raman spectroscopy or surface enhanced Raman spectroscopy (SERS). Samples comprising the bacteriophage, bacteria, and the complex are introduced to a solid support comprising a metal surface. In some embodiments, the metal surface comprises nanostructures. In some embodiments, the metal surface comprises nanoparticles arranged on the solid support by self-assembly. In some embodiments, the metal surface comprises functional groups for immobilizing the bacteriophage, bacteria, and/or the complex. In some embodiments, optical excitation of the samples can be carried out at 532 nm. The corresponding Raman shift from the sample can be measured by detecting the emission from the sample by a spectrometer. In some embodiments, Raman spectroscopy or SERS can be carried out in aqueous environment.

In some embodiments, the bacteriophages are detected by SPR. In some embodiments, the bacteriophage or the bacteria are immobilized on flow cells of a sensor surface. When a sample comprising the other partner (i.e. bacteriophage or the bacteria) which is not immobilized and free in solution, passes through the flow cell, the resulting complex is immobilized on the flow cell and the resulting signal is detected. In some embodiments, the sample flows on the flow cell at a constant rate.

In some embodiments, the bacteriophages are detected by electron microscopy. In some embodiments, the solid support comprises silicon nitride. In some embodiments, the solid support comprises functional groups to immobilize the bacteriophage and/or the bacteria. In some embodiments, the sample comprising bacteriophage and/or bacteria are introduced to the solid support by a flow cell. In some embodiments, a contrast reagent is added to the sample comprising bacteriophage and/or bacteria. Non-limiting examples of contrast reagent include uranyl formate, sodium phospho-tungstate, ammonium molybdate, methyl amine tungstate. In some embodiments, the transmission electron images are recorded on a CCD camera with magnification greater than 1000×.

In some embodiments, the bacteriophages are detected by scanning probe microscopy. In some embodiments, the detection is by atomic force microscopy. In some embodiments, the sample comprising bacteriophage, bacteria and/or the complex are introduced to the solid support. In some embodiments the bacteriophage, bacteria and/or the complex are immobilized on a solid support. In some embodiments, the solid support is glass. In some embodiments, the solid support may further comprise metal.

In some embodiments, the scanning probe is operated in constant height mode, deflection mode, tapping mode, or lift mode. In some embodiments, the morphology of the sample is recorded as height, amplitude or deflection of the probe. In some embodiments, the phage infection is deduced from the morphology data by comparing it with uninfected sample. In some embodiments, the scanning probe tip is biased by applying an alternate voltage or a direct current voltage. In some embodiments, a map of the contact potential of the phage infected bacteria is compared to the uninfected bacteria to evaluate susceptibility. In some embodiments, the probe tip comprises a thermal sensor. In some embodiments, the thermal sensor is a metal wire. In some embodiments, the probe tip forms one leg of a Wheatstone bridge. In some embodiments, the bridge is used to maintain the scanning probe tip at to measure the temperature of the scanning tip probe, or to maintain the tip at a constant temperature. In some embodiments, the heat flow from the sample is measured by at least one tip. In some embodiments, the heat flow from the sample measured by scanning at least one tip over the sample in the temperature contrast mode by measuring the resistance change of the probe. In some embodiments, the heat flow from the sample is measured in the current contrast mode by supplying additional energy to the circuit. In some embodiments, the energy required to maintain the constant current within the tip is a measured as the heat flow from the sample. In some embodiments, the difference in the heat flow from the sample to that of a negative control that does not contain the infectious phages is used to determine the infectious phages in the sample.

In some embodiments, the bacteriophages are detected by electrochemical device. In some embodiments, the device comprises of at least one fluidic chamber or channel comprising at least two electrodes and at least one fluidic connection between the electrodes. In some embodiments, the device comprises one or more sensors. In some embodiments, the bacteriophage, bacteria, and/or the complex are immobilized on a solid support of the device. In some embodiments, sample comprising the bacteriophage, bacteria, and/or the complex are flowed at a constant rate over the sensor of the device.

In some embodiments, the electrical current is measured as a function of the applied voltage to the electrodes. In some embodiments, the device measures the membrane potential in which a change in the membrane potential is indicative of phage infection of a bacterial cell. In some embodiments, the device measures the redox potential of a molecule. In some embodiments, the pH change is measured. In some embodiments, the enzyme activity is measured (NAD-NADH, ADP-ATP). In some embodiments, voltage sensitive dyes are used to measure response of the bacterial cells to phage infection. In some embodiments, the fluorescence spectrum of a dye is monitored as a function of membrane potential change.

In some embodiments, application of an electric field between the electrodes allows the differential migration of the bacteriophage, bacteria, or the complex of the bacteriophage and the bacteria to the positive electrode. In some embodiments, the device may comprise a sensor to detect the differential migration of the bacteriophage, bacteria, or the complex of the bacteriophage and the bacteria. In some embodiments, a direct current is applied. In some embodiments, an alternate current is applied.

In some embodiments, the bacteriophages are detected by change in mass. In some embodiments, the bacteriophage and/or the bacteria is immobilized on a solid support. In some embodiments, the immobilization is by a covalent bond between the functional groups and the bacteriophage, bacteria, and/or the complex. In some embodiments, the immobilization is by a non-covalent means, e.g., binding of a binding pair, e.g. biotin/streptavidin.

In some embodiments, the solid support comprises a sensor. In some embodiments, the sample comprising the bacteriophage, bacteria, and/or the complex is flowed over the sensor. In some embodiments, the bacteriophages are measured as a change in the mass upon phage complex formation. In some embodiments, the change in the mass on the sensor is detected by measuring the change is the resonance frequency of the sensor. In some embodiments, the sensor comprises a crystal. In some embodiments, the change is the mass of the sensor is derived by measuring the dissipation of shear movement of the crystal.

In some embodiments, the sensor is a piezoelectric sensor. In some embodiments, the piezoelectric sensor has at least a monolayer of metal, e.g., gold, silver, platinum etc. on top or bottom of the sensor surface. In some embodiments, the sensors are round-shaped. In some embodiments, the sensor is a quartz crystal with gold coating and mounted on a flow cell.

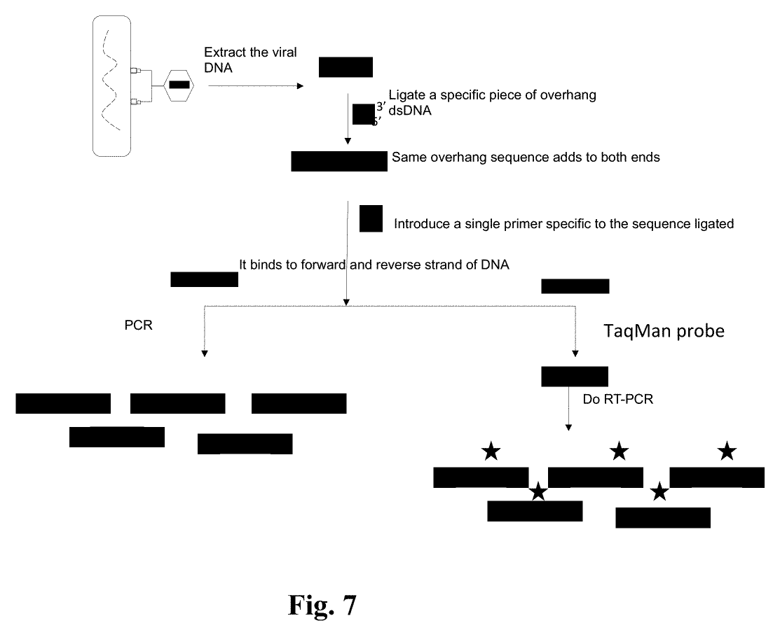

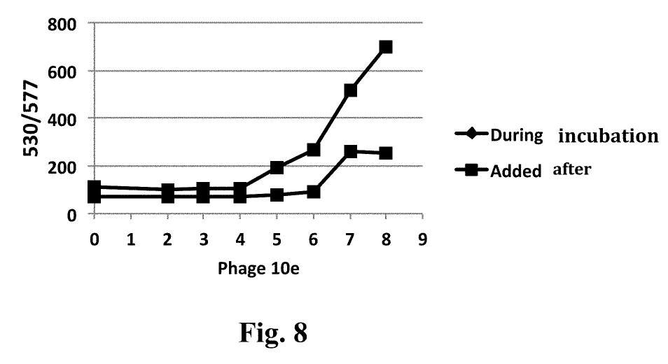

In some embodiments, the bacteriophages are detected by detecting the bacteriophage nucleic acid. In some embodiments, the complexes are separated from the incubation mixture of the bacteriophage and the bacteria. As shown in FIG. 7 and FIG. 8, the nucleic acid of the complex is isolated. The isolated nucleic acid will comprise bacterial nucleic acid and nucleic acid from the bacteriophages that specifically infect these bacteria. Bacterial DNA is large and supercoiled and plasmid DNA is supercoiled. In contrast, DNA from bacteriophage family Caudovirales is linear dsDNA. Thus, bacteriophage DNA can be separated from bacterial DNA due to the difference in size and morphology. The genomic DNA of the phages often contains staggered ends. Bacteriophages DNA ends are blunted. A double stranded DNA containing a known sequence that is referred herein as identifier sequence is treated with a restriction enzyme to produce a single staggered cut on the 5′-end. The identifier sequence is then ligated to the blunt ended phage DNA. A standard PCR reaction utilizing primers that recognize the unique identifier sequence is employed. This will amplify the phage DNA that was captured in the first step.

In some embodiments, the detection of bacteriophage nucleic acids is by mass spectrometry. Nucleic acid is extracted from a sample comprising bacteria and/or complex. The extracted nucleic acid is concentrated and applied on a solid support. The nucleic acid is dried. The mass spectra from the purified nucleic acid sample are recorded on a mass spectrometer device. The mass spectra of a standard healthy cells and phage infected cells are compared. A difference is indicative of phage infection.

In some embodiments, the bacteriophages are detected by using a calorimetric device. The endothermic and exothermic peaks of the negative control (uninfected bacteria) and the sample suspected of comprising a complex of bacteriophage and bacteria are obtained from thermograms of the calorimetry output. The peaks are compared to infer phage infection. In some embodiments, the onset temperatures of the endothermic or exothermic peaks are studied. In some embodiments, the total enthalpy of the sample is used to infer the presence of a phage infection.

In some embodiments, the bacteriophages are detected by fluorescence. In some embodiments, the bacteria and/or the bacteriophage comprise a detectable label. In some embodiments, the bacteria and/or the bacteriophage comprise more than one detectable label, e.g., at least two, at least three detectable labels. In some embodiments, the detectable labels are different. In some embodiments, the detectable label is a fluorescent moiety. In some embodiments, a fluorescent moiety can be stimulated by a laser with the emitted light captured by a detector. The detector can be a charge-coupled device (CCD) or a confocal microscope, which records its intensity.

In some embodiments, the bacteriophages are detected by the change in the physiological and chemical changes due to the complexation by enzymatic reaction. In some embodiments, the bacteriophages are detected by the color change due to enzymatic reaction, e.g., beta galactopyranoside assay, glucuronidase, N-acetyl-b-galactosidase, esterase, glucosidase, and lactate dehydrogenase (LDH).

### EXAMPLES

### Example 1: β-Galactosidase Activity of Bacteriophage Infected Lactic Acid Bacteria

The fermentation of milk into cheese and yogurt products relies on a class of Lactic Acid Bacteria (LAB). Despite extensive measures, phages are found to be the major cause of fermentation failure. Currently, the gold standard for phage detection is the double-agar plaque method. However, this method takes 24 hours, is user dependent and furthermore, negative results do not necessarily exclude phages as some can be difficult to visualize on agar plates. There is a need for more dependable and quick phage detection assays. Since the majority of cheese-making bacteria belong to the class of LAB, their metabolic pathways were utilized and enzymatic activities that are found in LAB were measured. The β-galactosidase activity of LAB has been extensively studied. Major efforts have been applied towards optimizing the release of intracellular enzymes such as the β-galactosidase from cells by sonication, chemical methods and vortexing with quartz sand (150 uM). Since host cells infected with phages are eventually lysed, releasing newly synthesized phages along with the bacteria's cellular contents, the measure and f-galactosidase activity were measured and correlated with phage infected cells.

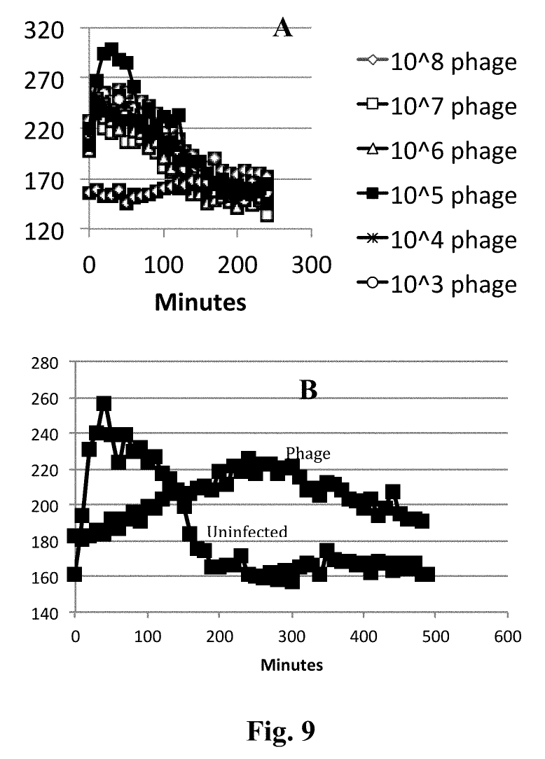

Results: FIG. 9 shows the results of the assay. Blue diamonds: S1 bacterial cells with 0.1 mM Res-Gal and phages were incubated at 37° C. overnight. The cell free extracts was obtained by centrifugation and measured for β-galactosidase activity. Cells infected with 10⁵-10⁸ phages had measurable β-galactosidase activity.

Red squares: S1 bacterial cells were incubated with phages overnight. The cell free extracts were obtained by centrifugation. Res-Gal was added to 0.1 mM, incubated for 1 hour and measured for β-galactosidase activity. In this method, only cells infected with 10⁷-10⁸ phages had measurable β-galactosidase activity.

Conclusion: These initial results show that cell lysis by phages leads to cellular release of metabolic enzymes such as β-galactosidase. The metabolic activity of LAB can be indicative of the health status of bacterial cells. Without further optimization, the current method detects phage infection at 10⁵ phages on a standard fluorescent spectroscopic plate reader. Further optimization of buffer, optimal temperature, pH and the working Res-Gal concentration can bring the level of detection down to as low as 10³-10⁴ phages on a standard plate reader. The same sample prep will be measured on our photonic platform which is more sensitive than a standard plate reader. In addition, testing of other commercially available fluorescently labeled galactopyranosides such as FDG-gal, MU-gal, DDAO-gal and ONPG-gal will be performed. And to test other enzymatic activities such as glucuronidase, N-acetyl-b-galactosidase, esterase, glucosidase, and lactate dehydrogenase (LDH).

### Example 2: Resazurin-Resorufin. A Live Cell Assay

During the production of cheese, a liquid protein by-product known as whey is collected and often reused as starter cultures for the fermentation of milk. However, a major source of phages is often found in downstream whey samples. Upon consultation with industry cheesemakers, they have noted that the presence of phages in the whey does not always result in failed cheese processing. In efforts to reduce waste they have expressed interest in risk assessment methods. A quick and simple assay using Resazurin can serve as a qualitative test for whether to continue using the whey. Resazurin is a weakly fluorescent compound that is sold as a cell viability assay. Only live cells can maintain a reducing environment that reduces the Resazurin compound to the highly fluorescent Resorufin. Lactic acid bacteria that have been infected with phages have a diminished or decreased ability to reduce Resazurin.

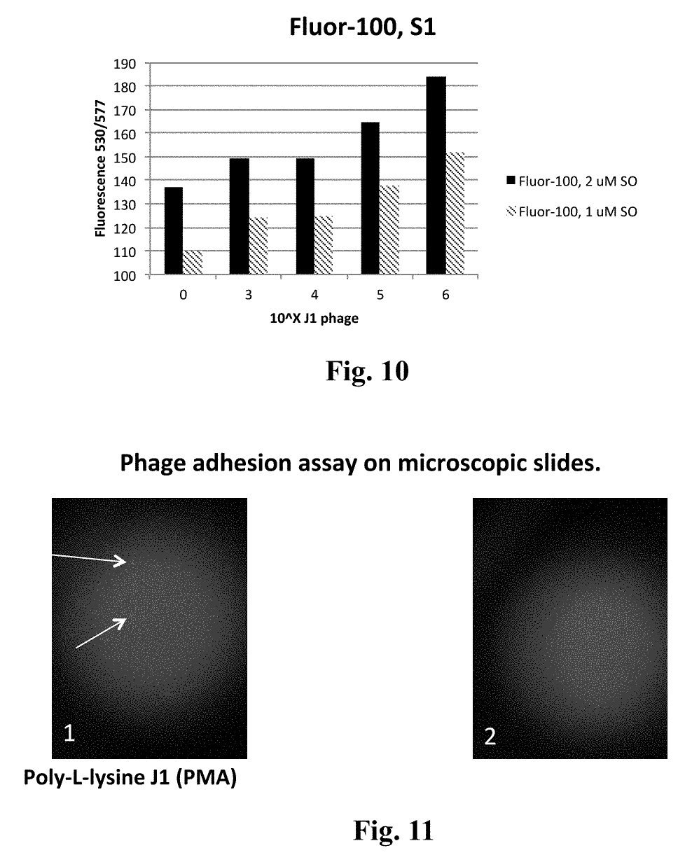

Results: Inventors have unexpectedly found that by comparing the kinetic profiles of phage infected cells versus healthy cells can be associated with the viability of the LAB cultures. By monitoring the fluorescent compound Resorufin, inventors have found that kinetic profiles obtained in less than 1 hour that correlates to the viability of the lactic acid bacteria. Healthy cells appear to peak within 20-40 minutes of adding the Resazurin reagent, whereas phage infected cells have a lower peak (FIG. 10A). Cells that have lost significant viability have a delayed peak taking as long as 240 minutes to reach their peak (FIG. 10B). The kinetic profiles were surprising results because Resazurin is marketed as an end point assay rather than for kinetics. However, it appears that lactic acid bacteria can carry out a secondary reduction of Resorufin that leads to the decrease of the fluorescent signal following the peak, which produces a dependable kinetic profile for understanding the effect on phage infection.

Conclusion: Inventors have discovered a simple and fast, and under 1 hour live cell kinetic assay that can be carried out on a standard plate reader to access the viability of cell cultures. This is a useful assay to access the overall health of cultures before utilizing them for fermentation. This quick and simple assay serves as a qualitative check point by comparing starter cultures with spent cultures (from whey).

### Example 3. Phase Adhesion Assay

Phages infect specific bacterial strains or a narrow host range. This specificity relies on interactions between phage protein receptors and glycoproteins on bacterial cell surfaces in order for specific phage adhesion to occur.

Results as shown in FIG. 10 from the experiments demonstrate that known host cells can be used to capture phages on their cell surface. By labeling the bacteria and phages in the samples, one can discriminate the fluorescent signal differences from a non-infected cell versus host cells that have been loaded with phages.

### Example 4. Phase Adhesion Assay on Microscooic Slides

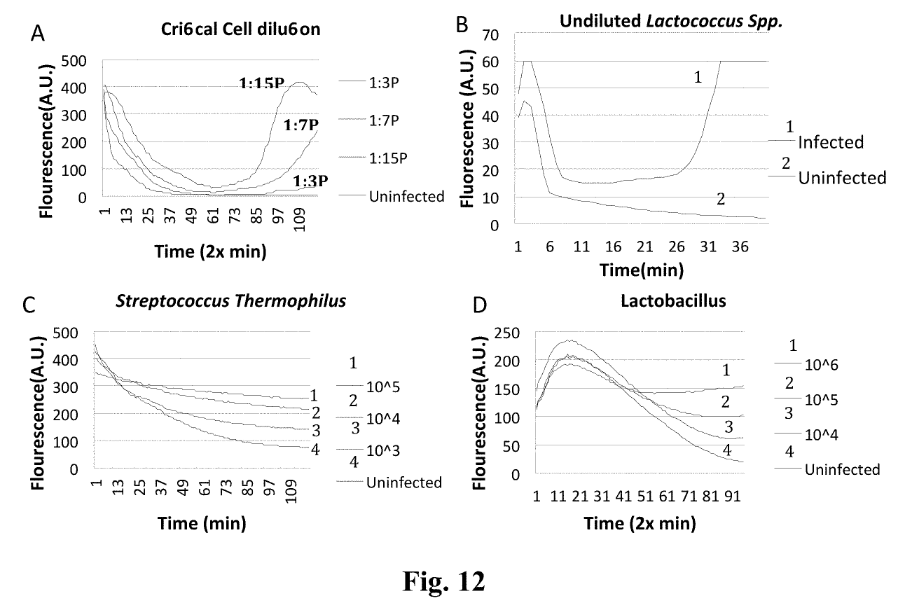

To further expand the assay for different platforms such as a fluorescent microscope, microscopic glass slides were treated with poly-L-lysine to promote the orientation of phages or promote tighter binding of bacteria on the surface of the slides. Sample containing a virulent J1 phage was added to the glass substrate with Poly-L-Lysine coating. The virulent phage was treated with Propidium Mono Azide (PMA™). The susceptible S1 bacteria from a starter culture were stained with SYBR® is introduced to the glass slide (FIG. 12-1). The arrow shows the binding of the bacteria to the slide. The arrow is pointing to very small rod-shaped bacteria as expected under 40× magnification.

Bacteria from a starter culture stained with SYBR® were introduced to the slide that does not have a virulent phage (FIG. 11-2).

### Example 5: Raman Spectroscopy and Surface Enhanced Raman Spectroscopy (SERS) for Bacteriophage Detection

For SERS internal mode, harvested bacterial cells are resuspended in AgNO3 solution (1 M) and incubated for 5 min. The cells are then collected and are washed twice in water by centrifugation at 4,500 g for 10 min at 4° C. Afterwards, the cells are resuspended in NaBH4 solution (0.5 M) for further analysis. For SERS external mode, harvested bacterial cells are first resuspended in NaBH4 solution and incubated for 5 min. The cells are then collected by centrifugation and resuspended in AgNO3 solution for further analysis. In a typical experiment, the bacterial cells are treated with 0.1% Triton X-100 (v/v) for 5 min, prior to the assay. Then, 10 μL of such treated bacterial suspension (˜1×106 CFU/mL) are mixed with 10 μL of NaBH4 (0.5 M). The mixture is then incubated for 3-5 min. Subsequently, 800 μL of AgNO3 (1 M) are pipetted into the mixture followed by vortexing.

Raman analysis is conducted by addition of 5 μL of the bacterial suspensions obtained from above modes onto the surface of glass cover slips (0.13 mm thickness, 15 mm diameter, Ted Pella Inc., Redding, Calif., USA). SERS spectra are recorded on a benchtop Raman spectrometer (Sierra Snowy Range 785 series, Laramie, Wyo., USA) under excitation wavelength of 785 nm. The exposure time is 1s and the number of accumulations for each measurement is 10. The spectral data are acquired over a Stokes Raman shift of 400-2,000 cm−1. For each study, three biological replicates are analyzed. To validate the results of the strain level discrimination, additional replicate is also analyzed.

### Example 6: Electrochemical Detection of Bacteriophage

Protocol

Functionalization of Electrochemical Sensors

Thiolated capture probe is prepared at a concentration of 0.05 μM in 300 μM 1,6-hexanedithiol (HDT), 10 mM Tris-HCl, pH 8.0, 0.3 M NaCl, 1 mM EDTA and is incubated in the dark at room temperature for 10 min. Incubation of the thiolated capture probe with HDT ensures that the thiol group on the capture probe is reduced, resulting in more consistent results.

A stream of nitrogen is applied to bare gold 16 sensor array chip(s) for 5 sec to remove moisture and/or particulates. A 6 μl of the HDT-thiolated capture probe mix is applied to the working electrode of all 16 sensors of the sensor array and store the sensor chip(s) in a covered Petri dish at 4° C. overnight. Thiolated capture probes bind directly to the bare gold electrode and the HDT acts to prevent overpacking of the capture probes and keep them in an extended conformation that promotes hybridization with the target.

The following day, the sensor chip is with deionized H2O for 2-3 sec and dry under a stream of nitrogen for 5 sec. A 6 μl of 10 mM Tris-HCl, pH 8.0, 0.3 M NaCl, 1 mM EDTA, 1 mM 6-mercapto-1-hexanol (MCH) is applied to the working electrode of all 16 sensors and incubate for 50 min. This and all subsequent sensor chip incubations are performed in a covered Petri dish at room temperature. MCH acts as a blocking agent, filling in any gaps where the thiolated capture probe or HDT is not present on the electrode surface.

Sample Preparation

One ml of bacterial culture is transferred in the log phase of growth (OD600=0.1) to a microcentrifuge tube and centrifuge at 16,000×g for 5 min. The culture supernatant is removed. The bacterial pellet can be processed immediately or can be stored at −80° C. for later use. The bacterial pellet is thoroughly resuspended in 10 μl of 1 M NaOH by applying the pipette tip to the bottom of the microcentrifuge tube and pipetting up and down several times. Incubate the suspension at room temperature for 5 min. The bacterial lysate is neutralized by adding 50 μl of 1 M Phosphate Buffer, pH 7.2, containing 2.5% bovine serum albumin (BSA) and 0.25 mM of a fluorescein-modified detector probe. The neutralized lysate is incubated for 10 min at room temperature. Fluorescein-modified detector probes hybridize with bacterial rRNA target molecules.

Electrochemical Sensor Assay

The MCH is washed from the sensor chip with deionized H2O for 2-3 sec and is dried under a stream of nitrogen for 5 sec. A 4 μl of neutralized bacterial lysate is applied to the working electrode of each of 14 sensors and incubate for 15 min. Target-detector probe complexes hybridize to immobilized thiolated capture probes. A 4 μl of 1 nM bridging oligonucleotide in 1 M Phosphate Buffer, pH 7.2, containing 2.5% BSA and 0.25 μM fluorescein-modified detector probe is applied to 2 positive control sensors (used for signal normalization) and is incubated for 15 min. The sensor chip is washed with deionized H2O for 2-3 sec and is dried under a stream of nitrogen for 5 sec. A 4 μl of 0.5 U/ml anti-Fluorescein-HRP in 1 M Phosphate Buffer, pH 7.2, containing 0.5% casein is applied to the working electrode of all 16 sensors and is incubated for 15 min. The anti-Fluorescein-HRP binds to the immobilized fluorescein-modified detector probes. The sensor chip is washed with deionized H2O for 2-3 sec and dry under a stream of nitrogen for 5 sec. A film well sticker is applied to the surface of the sensor chip and load into the sensor chip mount. A 50 μl of TMB substrate is pipette onto all 16 sensors and close the sensor chip mount.

Amperometry and cyclic voltammetry measurements are obtained for all 16 sensors using the Helios Chip Reader. Amperometric current is proportional to the rate of TMB reduction on the sensor surface.

### Example 7: Detection of Bacteriophage DNA by Mass Spectrometry

Mass spectra are acquired using an Ultraflex I MALDI-TOF mass spectrometer (Bruker Daltonics, Bremen, Germany). Alternatively, a simpler MALDI-TOF instrument such as the benchtop Microflex (Bruker Daltonics) can be used without losing data quality. Measurements in linear positive ion detection mode are performed, using a Nd:YAG laser at maximum frequency of 66 Hz. Pulsed ion extraction (PIE) is set to zero. Acceleration voltage (ISI) is set to 20 kV. The mass range of spectra is from 2,000 to 20,000 m/z. The final resolution in the mass range of 7,000-10,000 m/z is optimized to be higher than 600 and absolute signal intensities are about 103. Automated spectrum acquisition is performed using the Auto Execute software with fuzzy control of laser intensity. At least 107 bacterial cells are required for high quality mass spectra. For reference spectra six spots on the MALDI target are measured. On each spot, four spectra with 10 times 100 laser shots are accumulated. Twenty spectra are stored for the reference spectra library. For identification spectra is acquired by accumulating 1000 laser shots in ten 100 shot portions.

Factors influencing the intensities of signal peaks comprise concentration and location of proteins in the bacterial cell and biophysical properties of proteins such as solubility, hydrophobicity, basicity, and compatibility with MALDI. In general, most of the proteins detected by MALDI protein bacterial profiling derive from highly abundant, basic ribosomal proteins.

Data analysis: Mass spectra are analyzed with Flex Analysis software 2.4 (Bruker Daltonics). The mass spectral input data can be listed in generic data formats such as the extensible markup language (XML) to make them independent from the hardware used. Spectra are pre-processed using default parameters for reference spectra libraries also known as call main spectra libraries (MSPs). A maximum of 100 peaks with a signal-to-noise (S/N) ratio of 3 are selected in the range of 3,000-15,000 Da. Afterwards the main spectra are generated as a reference using all spectra given for a single microorganism. In general, 75 peaks are picked automatically, which occur in at least 25% of the spectra and with a mass deviation of 200 ppm.

For the evaluation of mass spectra reproducibility, the spectra are loaded into the ClinProTools 2.1 software (Bruker Daltonics). Through this process mass spectra are firstly normalized before baseline subtraction, peak detection, realignment, and peak-area calculation are applied. The optimal settings resulted in an S/N ratio of 5, a Top Hat baseline subtraction with 10% as the minimal baseline width, and a 3-cycle Savitsky-Golay smoothing with a 10 Da-peak width filter. For the example shown in FIG. 3 the coefficient of variation (CV) of each of the individual peak areas is determined; 100 peaks are taken for intra run assessment detected in 18 measurements and 75 peaks for inter run detected in 5 biological replicates. The mean CV for all of the signals from the same replicate sample is calculated to provide a measure of intra- and inter-run reproducibility.

### Example 8: Detection of Bacteriophage by Atomic Force Microscopy

A Nanoscope IIIA AFM (Digital Instruments, Santa Barbara, Calif., USA) operating in contact mode in air is used to image cells and to measure interaction forces. The relative humidity was 50-60% and no a spring constant of k50.06 N/m (Digital Instruments). The radius of curvature of the AFM tip is approximately 50 nm. The Digital Nanoscope software (version 4.23) is used to analyze the topographic images of the surface, as well as the force-distance over the sample surface. During the force-distance measurements, the scanning rate in z-direction is maintained at 30 Hz. Each map of sample surface consisted of 64 3 64 grid points.

### Example 9: Detection of Bacteriophage by Calorimetry

Samples (10-15 mg) are weighed to & 0.01 mg, sealed in volatile aluminum pans and is heated in a Perkin-Elmer DSC-2C at a rate of 10° C. min⁻¹ from about 10° C. to 120° C. Samples are weighed before and after calorimetric measurements to check for loss of mass during heating and the results of samples showing signs of leakage are discarded. An empty pan is used as a reference and, after heating, the sample is rapidly cooled to its initial temperature. Selected samples are then re-run in the DSC to investigate the reversibility of the thermograms. The sample dry mass is determined by piercing the pan and drying it overnight in an oven at 105° C. Data are collected and the calorimeter is controlled with a Perkin-Elmer data station but thermogram scans are transferred to a VAX-l1/750 computer for high-resolution plotting and peak area determined by trapezoidal integration. The differences between the data collected during the first run and those collected on re-running the DSC are proportional to that component of the specific heat capacity caused by irreversible processes taking place during the first heating. The difference thermogram is useful for precise determination of the onset temperature of irreversible denaturation. Temperature and power scales are calibrated according to the manufacturer's instructions using the melting of indium and ice as standards.

### Example 10: Detection of Bacteriophage by Monitoring the Reduction of Resazurin and Resorufin

To 1-1000 μl of samples (milk, whey, salt whey, culture, milk culture, broth), 1-1000 μl of bacterial cells with OD˜0.1-1 were added. In some cases, bacteriophages specific for the bacteria are added. In some embodiments, the bacteriophages were bound by a binding agent. To this mixture, Resazurin and/or Resorufin and/or Dihydroresorufin were added as a fluorescent tag. The fluorescence of Resorufin was measured between 25-47° C. In some experiments, the sample was incubated with the cells before the addition of the fluorescent tag. In some samples, the fluorescent tags were incubated with the sample. In some experiments, fluorescent tags were incubated with cells. In some experiments, the incubated sample and/or cells were diluted before the addition of the tag. In some experiments, the incubated sample and/or cells were concentrated before the addition of the tag. In some experiments, the fluorescence of Resorufin was measured at 577 nm over a period of time. The kinetic profile of the reduction of Resazurin to Resorufin to Dihydroresorufin of bacterial cells infected with bacteriophage were compared to corresponding uninfected standard bacterial samples. The above conditions were tested in the following experiments.

1. Incubating Cultures and Bacteriophages Together at 32° C., Diluting Samples and/or Bacteria and Adding Resazurin to the Samples

In one case, wild-type phages and cocci and/or bacillus (gram positive) bacterial culture were added to milk. The mixture was incubated for 45 min. Resazurin was added and mixed. The fluorescent intensity of the mixture was measured at 577 nm for at least 30 min and shown in FIGS. 12A and 12B.

In another case, wild-type phages and cocci and/or bacillus bacterial culture were added to of bacterial culture medium. The mixture was incubated for 90 min. To the incubation mixture, Resazurin was added and mixed. The absorbance and/or absorbance, scattering and/or fluorescent intensity of the mixture was measured at 577 nm for at least 20 min. Longer incubation times (75-120 minutes) resulted in the uninfected cells converting more of the Resorufin to Dihydroresorufin thus the infected cells had higher levels of fluorescence and the lines cross over.

In another case, wild-type phages and cocci and/or bacillus bacterial culture were added to bacterial culture medium. The mixture was incubated for 75 min. To the incubation mixture, Resazurin was added and mixed. The absorbance and/or absorbance, scattering and/or fluorescent intensity of the mixture was measured at 577 nm for at least 40 min. Longer incubation times also increased sensitivity allowing the detection of bacteriophage as low as 10³ pfu/mL.

Additionally, inventors identified an unexpected and surprising difference in the kinetic profile of the reduction of Resazurin to Resorufin to Dihydroresorufin between phage infected bacterial cells and uninfected bacterial cells. The uninfected bacterial cells reduced the Resazurin (weakly fluorescent) to Resorufin (fluorescent) and finally to Dihydroresorufin (non-fluorescent) at a faster rate as compared to phage infected bacterial cells. The reduction of Resazurin to Resorufin is irreversible while the reduction of Resorufin to Dihydroresorufin is reversible. After some time, the predominant metabolic product of Resazurin is Dihydroresorufin (non-fluorescent) for uninfected bacterial cells. As a result, the fluorescence peaks faster for uninfected bacterial cells, and the fluorescence diminishes with time.

In contrast, the reduction of Resazurin (weakly fluorescent) to Resorufin (fluorescent) is slower for phage infected bacterial cells. As a result, the fluorescence peak for Resorufin appears later in phage infected bacterial cells. Additionally, phage infected bacterial cells preferably oxidizes Dihydroresorufin (non-fluorescent) to Resorufin.

Difference in Time to Detection of Phage with Different Bacteria and Phages.

The time to detection of phage infected bacteria varied with different cells and phage. cocci and/or bacillus cells and wild-type phage required almost twice as long for the assay to detect phage infected cocci and/or bacillus bacteria as compared to different wild-type phage Detection of wild-type phage infection of the cocci and/or bacillus bacteria culture was also faster at 37° C. than 32° C. as shown in FIG. 12 B-D.

2. Adding Resazurin to the Cells Immediately Instead of after Incubation

Adding Resazurin at the start of incubation (TO) resulted in an increased efficiency of the assay by reducing a step. The infected cells having higher levels of fluorescence compared to the uninfected control. All samples rapidly become pink as the Resazurin is reduced irreversibly to Resorufin. The sample then became white in color as the Resorufin is reduced to Dihydroresorufin, a reversible step. After this step occurs, based on the phage concentration, phage infected cell samples start to have an increased pink color (Resorufin) while readings on the uninfected controls continue to decrease.

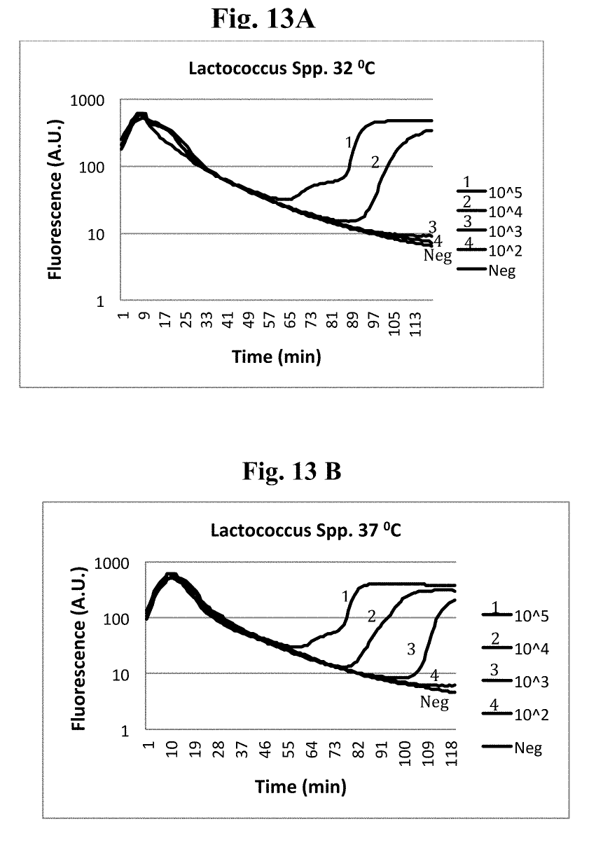

When varying concentration of wild-type phages (10⁵ pfu/ml-0 pfu/ml), cocci or bacillus bacterial culture and Resazurin was added and mixed with milk and incubated for 4 hours, the kinetic profile of the reduction of Resazurin was significantly different than of the phage infected cells. The data as shown in FIG. 13 A.

3. Effect of Temperature on the Length of Time Required to Detect Infection of Bacteria with Phages.

FIG. 13 A and B show the effect of temperature on the limit of detection of bacteriophage. In some experiments, higher temperature increases the limit of detection. In some experiments, lower temperature increases the limit of detection.

4. Difference Between Starting Incubations with Resorufin and Resazurin.

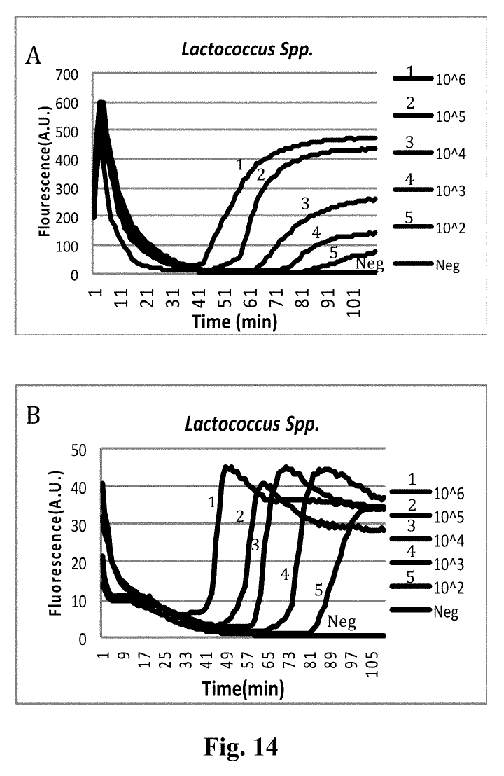

To see if the times to detect the bacteria could be decreased further, Resorufin instead of Resazurin was added to eliminate the time required for the cells to convert Resazurin to Resorufin. This did not significantly shorten the length of time to detection of phage infected bacteria using phage at higher concentrations but did shorten times by about 5-10 minutes for lower concentrations of virus (FIG. 14A-B). When the initial compound is Resazurin the fluorescence and/or absorbance intensity of Resorufin shows a correlation with the phages. It is observed that in addition to the change in kinetics of change from Dihydroresorufin to Resorufin in the presence of infectious phages, the amount of Resorufin oxidized from Dihydroresorufin is proportional to the phage concentration when the reagent used is Resazurin. When the initial reagent is Resorufin, in some experiments, the final oxidized fluorescence shows little correlation to the amount of phage present.

5. Detection of Bacteriophage from a Blend of Bacteria Added to the Sample.

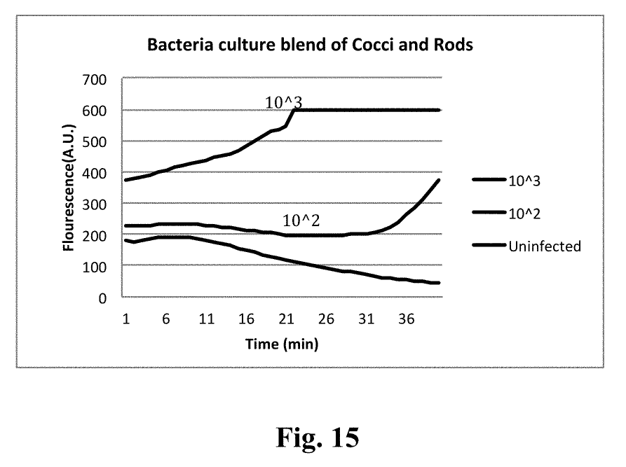

Bacteriophage susceptible to one or more bacteria in a blend of bacteria were detected (FIG. 15). This result shows that the Resazurin and/or Resorufin and/or Dihydroresorufin can be used to test multi-strained cultures.

6. Detection of Bacteriophage from Cultures Grown Fresh and from Thawed Frozen Cultures.

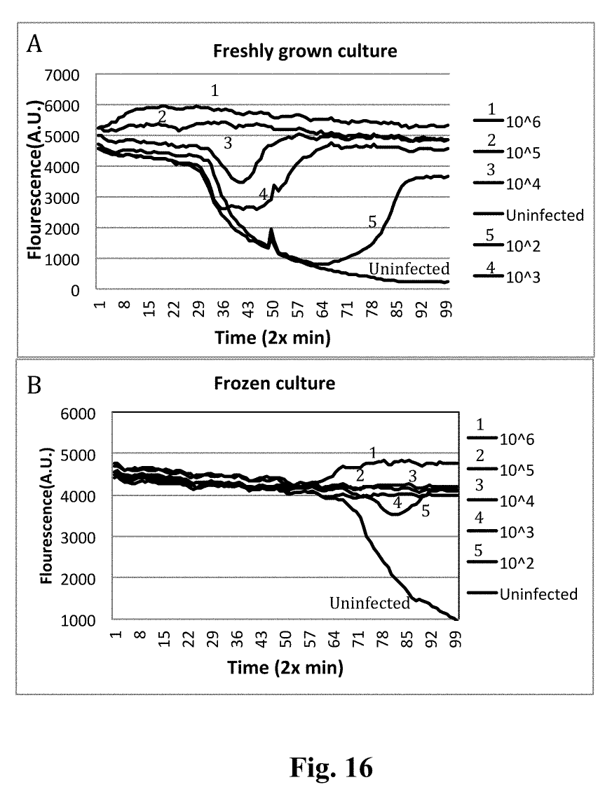

The effect of the cultures grown fresh versus frozen is shown in FIGS. 16 A and B. The kinetic feature of the frozen culture is observed to change with different thawing temperatures and time.

### Example 11: Detection of Bacteriophages by Preferential Labeling of DNA with Nucleic Acid Binding Dyes

A bacterial based assay was developed to capture host-specific bacteriophages. The captured bacteriophages are stained with SYTOX® Orange (Molecular Probes) and SYTOX® Orange has fluorescence enhancement of >500-fold upon binding DNA. SYTOX® orange is a cell impermeant nucleic acid dye that stains compromised cells. In every batch of healthy cells, there exist a percentage of dead cells due to the natural life and death cell cycle. This causes unpredictable background fluorescence due to dead cells.

To address the dye entering normal dead cells found in all bacterial samples, a protocol was developed using commercially available nucleic acid stains to preferentially block the signal from compromised bacteria and allow us to only detect bacteriophage.

Propidium monoazide (PMA, Biotium, Fremont, Calif.) (AB/Em, 510/610) is a cell impermeant, fluorescent dye that forms covalent bonds to nucleic acids of compromised cells. By strategically staining the bacteria-phage complex, bacteriophages can be detected specifically.

Bacterial cells were washed with PBS to remove media and cell debris before staining with PMA. The PMA dye preferentially stains compromised bacterial cells and is classified as cell impermeant. The bacterial cells are used as the capturing vehicle to attract host specific bacteriophages. Non-specific bacteriophages do not bind to bacteria and are washed away after pelleting the bacteria and a wash step.

SYTOX® orange or another similar cell impermeant dye is used to stain the nucleic acids of the bacteriophages. PMA continues to block the SYTOX® orange from intercalating with DNA in compromised bacterial cells. Therefore, the total signal output is from captured phages.

Briefly, 0.5 ml of bacterial cells, (OD 0.1-0.9) centrifuged in 1.5 ml tube for 13k rpm for 1 min. The supernatant was removed and the cell pellet was resuspended in 1 mL buffer to wash the cells. The wash step was repeated to remove the media and cell debris. 5-10 uL PMA (1-10 mM) was added to the washed cells and incubated in the dark for 5 minutes. The sample was exposed to light for 15 minutes to allow the covalent linking of PMA to the nucleic acid. The PMA labeled bacteria were ready to be used as the capturing vehicle of host-specific phages. Following the capture of the specific bacteriophages, 1-10 uM of SYTOX orange or another cell impermeant dye was used to stain the captured phages. The fluorescent signal was detected to detect the captured bacteriophages. The results are shown in FIG. 10. Bacteriophages at a concentration of 10³-10⁴ pfu/μl. Higher signals are observed when bacteriophages at a concentration of 10⁴-10⁵ pfu/μl were used.

### Example 12: Immobilization of a Complex of Bacteriophage and Bacteria on Beads

Preparation of S1 Host Bacteria

Bacterial host cell, S1 was washed twice with 1×PBS pH 7.4 buffer to remove MRS media. SYBR® green (Invitrogen) stain was added to a final concentration of 2×.

Preparation of PMA Stained J1 Phage

One mL of J1 phages (10⁸ PFU/ml) were concentrated using 50 kDa Amicon ultrafiltration filter (EMDmillipore, Billerica, Mass.). Spins were conducted at 7000 rpm for 5 minutes on a tabletop centrifuge. J1 phage was recovered in 400 μL. To the concentrated J1 phage, 2.5 μL of 20 mM PMA stain was added.

Preparation of Beads

200 μL of BioMag (51 mg/ml) amine beads (Bangs Laboratory, Fishers, Ind.) were washed with 1×PBS pH 8.0 buffer. Beads were resuspended in final volume of 200 μL 1X PBS pH 8.0 and served as the stock beads for the following experiment.

To prepare BS3 (bis(sulfosuccinimidyl)suberate), (Thermo Fisher Scientific) linked magnetic beads, 5 μl of stock beads was dispensed into 200 μL of 1×PBS pH 8.0. Then, 20 μL of 70 mM solution of BS3 was added to the beads and allowed to incubate for 45 minutes at room temperature on a rotating wheel. The BS3 linked beads were equally split into two 100 μL portions and then placed on a magnetic rack for 3 minutes. The solution containing excess BS3 linker was removed from the beads. To attach J1 phage to the linked beads, 200 μL of 109 PFU/ml J1 stained with PMA was added to one sample of beads and incubated at room temperature for 50 minutes on a rotating wheel.

The control set were non BS3-linked magnetic beads and these were prepared by dispensing 5 μL of stock magnetic beads into 200 μL 1×PBS pH 8.0. The sample was split into two 100 μL portions and placed on the magnetic rack to remove the buffer. J1 (200 μL of 10\hat{ }9 PFU/ml) stained with PMA was added to one of the beads.

Excess and unreacted linkers were quenched by adding 10 μL of 1M Tris HCl pH 7.5 to all four samples and incubated for 15 minutes at room temperature. The samples were placed on a magnetic rack to remove the solution.

500 μL MRS was added to all 4 samples to block remaining open sites on the bead for 1 hour.

Beads were washed twice with 1×PBS and ready to use.

Addition of S1 to Prepared Magnetic Beads

The SYBR® green stained S1 bacteria (200 μL of 108 cells) was added to each of the four bead samples and incubated with for 5 minutes at room temperature. The beads were then washed four times with 500 μL 1×PBS, pH 7.4 to remove unbound bacteria and resuspended in a final volume of 90 μL 1×PBS.

Results:

Non-BS3 linked magnetic beads with passive adsorption of J1 (PMA) onto the magnetic beads: Did not show signs of bacteria binding to the beads (FIG. 3, Col. B, Row (i) (fluorescence image) and Row (ii) (bright field image)).

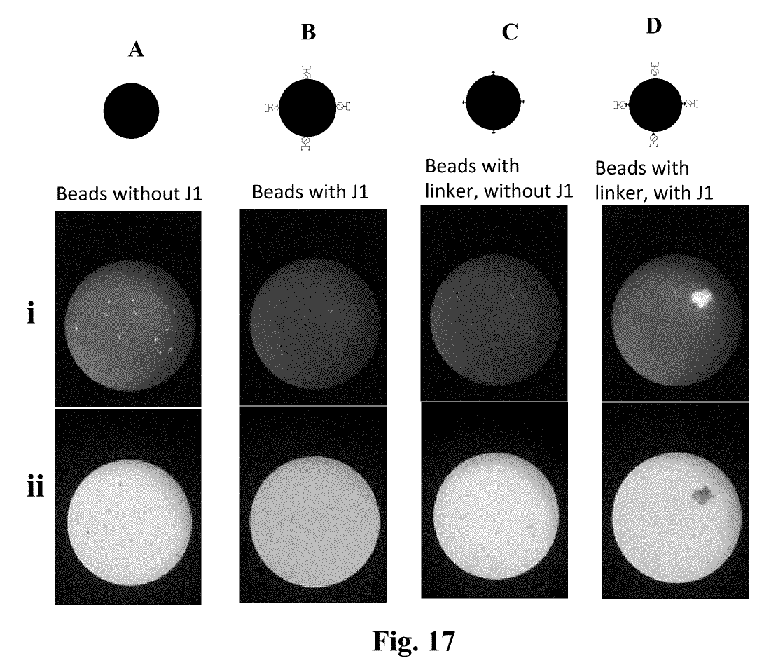

Non-BS3 linked magnetic beads without exposure to bacteriophage J1 (PMA stained) had low levels of S1 bacteria binding to the beads. Most of the beads were without bacteria (FIG. 17, Col. A, Row (i) (fluorescence image) and Row (ii) (bright field image)).

BS3-linked magnetic beads comprising the linkers but without immobilizing bacteriophage J1 (PMA stained) had very low levels of S1 bacteria bound to the beads. Most of the beads were without bacteria (FIG. 3, Col. C, Row (i) (fluorescence image) and Row (ii) (bright field image)).

Bacteriophage J1 (stained with PMA) immobilized to magnetic beads with the BS3-linker showed the most bacteria bound compared to the above three samples. Even though most individual beads were not coated in bacteria, there were clumps of magnetic beads that had bound bacteria (FIG. 17, Col. D, Row (i) (fluorescence image) and Row (ii) (bright field image).

### Example 13: Preferential Labeling of DNA with Nucleic Acid Binding Dyes

The preferential labeling of nucleic acid was tested with propidium monoazide (PMA), propidium iodide (PI) and SYTOX® Orange dyes. Permeable bacterial cells were initially stained with one of the three dyes and then stained with the other two dyes individually to test the blocking of the nucleic acid or displacement of the dyes from the nucleic acid.

0.6 ml of permeable S1 bacterial cells (10 s cfu/ml) were centrifuged at 13000 rpm. The supernatant was removed and the pellet was resuspended in 1 mL PBS. This wash step was employed to remove the media and cell debris.

In one set, 1-15 μL PMA (10 mM) was added to the washed cells and incubated in the dark or under light for 5-15 min. In some cases, the sample was exposed to light for 15-30 minutes. The bacterial cell solution was equally divided into two parts. To each bacterial cell solution, SYTOX® Orange (10 mM) and PI (10 mM) were added, respectively. The fluorescence was measured at different wavelength of excitation and emission corresponding to SYTOX® Orange, PI and PMA dyes to observe the effect of the second dye on the presence of PMA bound to the nucleic acid.

In another set, SYTOX® Orange (10 mM) was added to the washed cells and incubated in the dark or under light for 5 min. The bacterial cell solution was equally divided into two parts. To each bacterial cell solution, of PMA (10 mM) and PI (10 mM) were added, respectively.

In another set, PI (10 mM) was added to the washed cells and incubated in the dark or under light for 5-15 min. The bacterial cell solution was equally divided into two parts. To each bacterial cell solution, PMA (10 mM) and SYTOX® Orange (10 mM) were added, respectively.

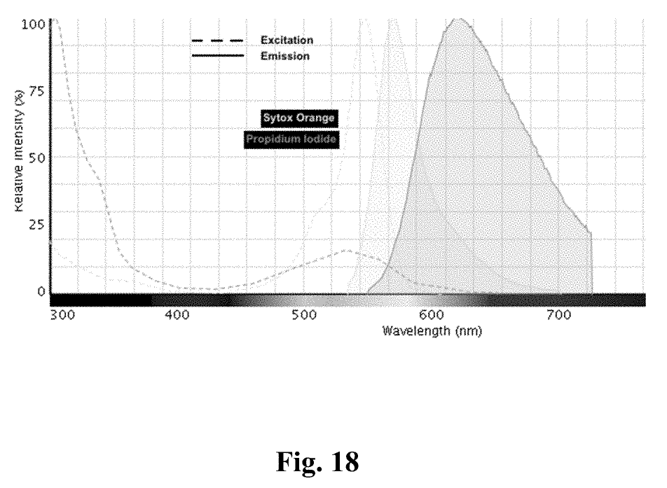

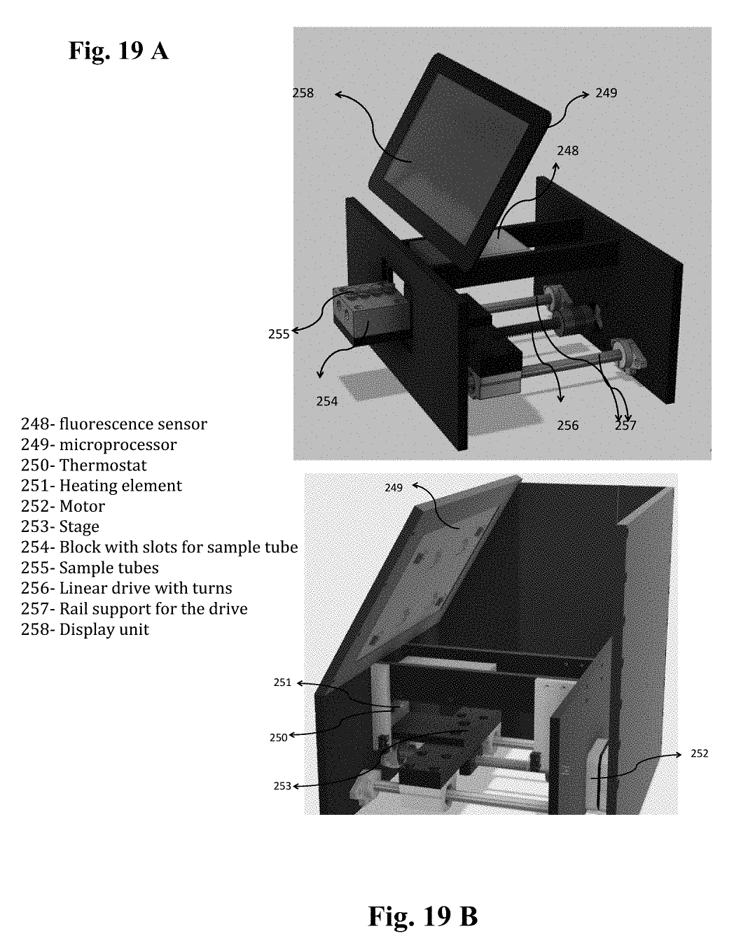

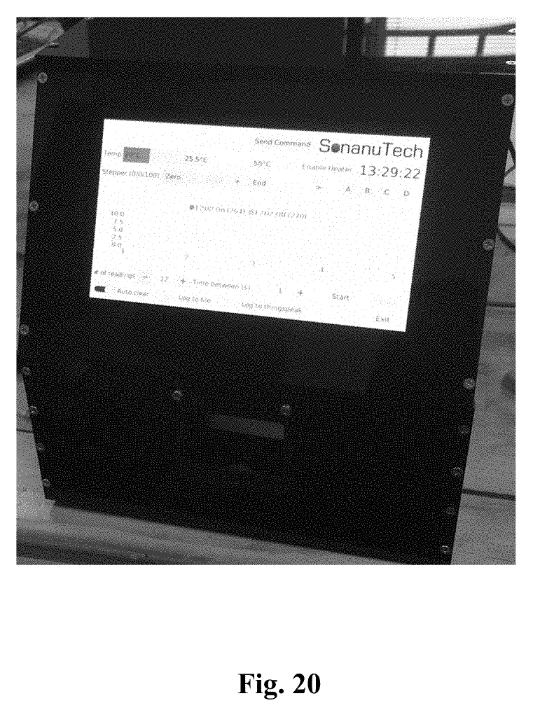

The fluorescence was measured at different wavelength of excitation and emission corresponding to SYTOX® Orange (530/577 nm), PI (492/600 nm, 20 nm emission bandwidth) and PMA (492/600 nm, 80 nm emission bandwidth) dyes to observe the effect of the second dye on the presence of PMA bound to the nucleic acid. Exemplary excitation and emission spectra of SYTOX orange and propidium iodide dyes are shown in FIG. 18. The results are shown in Table 3 below.

TABLE 3
Blocking and Displacement of the Dyes

| | Excitation/Emission | | | |
|---|---|---|---|---|
| | Wavelengths | | | Observation |
| S1 without stain | 16 | 33 | 46 | |
| S1 with SYTOX ® | 1514 | 288 | 816 | leak of |
| orange (SO) | | | | SYTOX ® |
| | | | | signal into PI |
| | | | | and PMA |
| Add PMA to the | 21 | 58 | 137 | PMA added |
| SYTOX ® stained | | | | after staining |
| S1 | | | | with |
| | | | | SYTOX ® |
| | | | | PMA |
| | | | | displaces |
| | | | | SYTOX ® |
| | | | | decreasing the |
| | | | | fluorescence |
| | | | | of SO and |
| | | | | increasing |
| | | | | PMA |
| | | | | fluorescence. |
| | | | | Low signal in |
| | | | | PMA channel |
| | | | | is because |
| | | | | efficiency of |
| | | | | PMA is low |
| Add PI to the | 143 | 136 | 300 | PI displaces |
| SYTOX ® stained | | | | SYTOX ® |
| dye | | | | |
| S1 with PI | 50 | 109 | 240 | |
| Add SYTOX ® | 158 | 139 | 317 | some |
| Orange to PI | | | | displacement |
| stained S1 | | | | but not |
| | | | | significant |
| Add PMA to PI | 22 | 58 | 150 | PMA |
| stained S1 | | | | displaces PI. |
| | | | | Fluorescence |
| | | | | of PI is |
| | | | | lowered with |
| | | | | PMA addition |
| S1 with PMA | 20 | 57 | 129 | |
| Add PI to PMA | 32 | 62 | 135 | No significant |
| Stained S1 | | | | change to |
| Add SYTOX ® | 31 | 48 | 126 | PMA |
| Orange to PMA | | | | fluorescence. |
| Stained S1 | | | | PMA is not |
| | | | | displaced by |
| | | | | PI or |
| | | | | SYTOX ® |
| | | | | Orange. The |
| | | | | PMA is a |
| | | | | strong |
| | | | | binder/blocker |

1. A method of detecting one or more types of bacteriophage in a sample comprising:
a) providing a first sample suspected of comprising said one or more types of bacteriophage;
b) providing a second sample comprising one or more types of bacteria, wherein said one or more types of bacteria are susceptible to infection by said one or more types of bacteriophage;
c) contacting said first sample with said second sample to form a sample mixture, wherein said one or more types of bacteriophage if present in said first sample forms one or more complexes with said one or more types of bacteria in said sample mixture;
d) contacting one or more compounds to said sample mixture;
e) measuring any change of said one or more compounds in presence of said sample mixture, wherein any change of said one or more compounds is indicative of the presence of one or more types of bacteriophage infected bacteria in said sample mixture, and wherein the presence of one or more types of bacteriophage infected bacteria in said sample mixture is indicative of the presence of one or more types of bacteriophage in said first sample.

2. The method of claim 1, further comprising measuring any change of said one or more compounds in presence of said sample mixture over a period of time and determining the kinetic profile of said change of said one or more compounds, wherein the kinetic profile is indicative of the presence of one or more types of bacteriophage infected bacteria in said sample mixture, and wherein the presence of one or more types of bacteriophage infected bacteria in said sample mixture is indicative of the presence of bacteriophage in said first sample.

3. The method of claim 1, wherein the identity of the one or more types of bacteriophage if present in said first sample are unknown.

4. The method of claim 1, wherein said first sample is selected from the group consisting of water, liquid collected from facility equipment, swabbed facility surfaces, fluid samples from facilities, fermented liquid, raw milk, pasteurized milk, skim milk, whey, starter culture, milk starter culture, whey starter culture and broth grown starter culture.

5. The method of claim 1, wherein first sample suspected of comprising one or more types of bacteriophage is incubated with said second sample suspected of comprising one or more types of bacteria susceptible to infection by said one or more types of bacteriophage prior to adding said one or more compounds.

6. The method of claim 2, further comprising comparing said kinetic profile of the change said one or more compounds with a kinetic profile of the change said one or more compound in the presence of one or more types of uninfected bacteria, wherein a difference between kinetic profiles is indicative of the presence of one or more types of bacteriophage in said first sample.

7. The method of claim 1, wherein said one or more compounds is an enzymatic substrate of said one or more types of bacteria.

8. The method of claim 1, wherein said one or more compounds is a redox compound.

9. The method of claim 1, wherein said one or more compounds comprise a detectable label.

10. The method of claim 9, wherein said detectable label is a fluorescent label.

11. The method of claim 8, wherein said one or more compounds is Resazurin, Resorufin, Dihydroresorufin, or a combination thereof.

12. The method of claim 11, wherein change of said one or more compounds comprise a change in the oxidative state of Resazurin, Resorufin, Dihydroresorufin, or a combination thereof.

13. The method of claim 12, wherein said measuring any change of said one or more compounds further comprises measuring the oxidative status of Resazurin, Resorufin, Dihydroresorufin or a combination thereof.

14. The method of claim 11, wherein said method is carried out at 32° C.

15. The method of claim 11, wherein said method is carried out at 37° C.

16. The method of claim 11, wherein said method is carried out at 40° C. or 42° C.

17. The method of claim 8, wherein measuring any change of said one or more compounds comprise measuring electrochemical signal.

18. The method of claim 8, wherein measuring any change of said one or more compounds comprise measuring electrical signal.

19. The method of claim 1, wherein said first sample is partitioned into solid phase and liquid phase prior to contacting with said second sample, and wherein said partitioned liquid phase of said first sample is contacted with said second sample.

20-41. (canceled)

42. A kit for detecting the presence of one or more types of bacteriophage in a sample using the method of claim 1, comprising:
a) a first reagent comprising one or more types of bacteria, wherein said one or more types of bacteria are susceptible to infection by said one or more types of bacteriophage, and wherein said one or more types of bacteria forms one or more types of complexes with said one or more types of bacteriophage if present in said sample;
b) buffers;
c) a second reagent comprising one or more compounds, wherein said compounds are differentially susceptible to change in the presence of one or more types of phage infected and uninfected bacteria;
d) instruction for forming complexes of said one or more types of bacteria with one or more types of said bacteriophage;
e) instruction for detecting any change to said one or more compounds, wherein a differential change in said one or more compounds for phage infected as compared to uninfected bacteria is indicative of the presence of said one or more types of bacteriophage in said sample.
